(19) United States
(12) Patent Application Publication
(10) Pub. No.: US 2025/0352076 A1
(43) Pub. Date: Nov. 20, 2025

(54) NOVEL COMPONENTS FOR A HEMODYNAMIC ANALYSIS TOOL

(71) Applicant: The Regents of the University of California, Oakland, CA (US)

(72) Inventor: Lawrence William Ang, San Diego, CA (US)

(21) Appl. No.: 19/029,975

(22) Filed: Jan. 17, 2025

Related U.S. Application Data
(63) Continuation of application No. PCT/US2023/071266, filed on Jul. 28, 2023.
(60) Provisional application No. 63/393,200, filed on Jul. 28, 2022.

(51) Int. Cl. A61B 5/021; A61B 5/00; A61B 5/02; A61B 5/026; G16H 50/20

(52) U.S. Cl. CPC A61B 5/021; A61B 5/02028; A61B 5/026; A61B 5/7239; A61B 5/7264; A61B 5/7271; G16H 50/20

(57) ABSTRACT

In some implementations, a method includes obtaining a set of time series ventricular pressure measurements; determining a set of data points comprising time rates of change of ventricular pressure from the time series ventricular pressure measurements; determining a representation indicative of a relationship between at least the set of data points and the time series ventricular pressure measurements; and determining a characteristic of blood flow within chambers of the heart at least in part by processing the representation. Related systems and articles of manufacture are also disclosed.

### CROSS-REFERENCE TO RELATED APPLICATIONS

This application is a continuation of Patent Cooperation Treaty Application No. PCT/US2023/071266 filed Jul. 28, 2023, entitled “NOVEL COMPONENTS FOR A HEMODYNAMIC ANALYSIS TOOL,” which claims priority to U.S. Provisional Application No. 63/393,200 filed on Jul. 28, 2022, entitled “NOVEL COMPONENTS FOR A HEMODYNAMIC ANALYSIS TOOL,” the disclosures of which are incorporated herein by reference in their entirety.

### BACKGROUND

Invasive measurement of ventricular blood pressure (Pv) and arterial blood pressure (Pa) is routinely performed in patients undergoing invasive evaluation in the cardiac catheterization laboratory. Pv measurements typically include 1) maximum systolic pressure, 2) minimum diastolic pressure, 3) end-diastolic pressure, 4) maximum rate of pressure change per time (dP/dt), and 5) continuous tracings visually displayed by existing commercial products as Pv versus time. These measurements are used by physicians to evaluate cardiac contractile (systolic) function, relaxation (diastolic) function, and diastolic filling pressure with the goal to identify and treat cardiac abnormalities.

### SUMMARY

In some example embodiments, there may be provided methods, systems, and articles of manufacture for analyzing characteristics of blood flow within the chambers of the heart and its downstream vessels as substantially described and shown herein.

In some embodiments, there is provided a system including at least one data processor and at least one memory storing instructions which, when executed by the at least one data processor, cause operations including obtaining a set of time series ventricular pressure measurements; determining a set of data points comprising time rates of change of ventricular pressure from the time series ventricular pressure measurements; determining a representation indicative of a relationship between at least the set of data points and the time series ventricular pressure measurements; and determining a characteristic of blood flow within chambers of the heart at least in part by processing the representation.

In some variations, one or more features disclosed herein including one or more of the following features may be implemented as well. The time series ventricular pressure measurements are collected using a device that is not inserted into a body of a subject. The device is a non-invasive ultrasound Doppler device, magnetic resonance imaging device, and/or cardiac sound intensity device. The time series ventricular pressure measurements are collected by an intracardiac device. The intracardiac device is a pulmonary artery hemodynamic monitoring catheter or a left ventricular support device. The time series ventricular pressure measurements are collected at least in part by measuring chamber dimensions and ventricular blood pressure. The relationship includes a set of pairwise relations, a pairwise relation comprising a data point of the set of data points and a corresponding time series ventricular pressure measurements. A time rate of change of ventricular pressure of the time rates of change of ventricular pressure is a first derivative of ventricular pressure with respect to time. The representation comprises a plot associated with the relationship. The plot is a pressure loop plot. The characteristic of blood flow is determined based at least in part on a loop cycle duration of the pressure loop plot. The characteristic of blood flow is determined based at least in part on a border of the pressure loop plot. The border is a top border, a bottom border, a left border, or a right border. The characteristic of blood flow is determined based at least in part on a visual characteristic associated with the visual plot. The visual characteristic is associated with a shape of a region of the visual plot or a size of at least a region of the plot. The visual characteristic is symmetry, smoothness, a presence of an indentation, a difference between two or more regions, or a tangential slope. The characteristic of blood flow is determined at least in part by comparing the visual plot with a second visual plot. A treatment regimen may be based at least in part on the characteristic of blood flow. The characteristic of blood flow is ventricular power, ventricular resistance, or ventricular blood flow, elasticity, compliance, contractility stroke volume, or response to a modifying factor. A second set of data points comprising time rates of change of acceleration of ventricular pressure may be calculated.

A pre-a wave diastolic pressure may be evaluated using at least in part the second set of data points. Processing the representation comprises using a mathematical model. The mathematical model is a statistical model or a machine learning model. The machine learning model comprises a neural network. A data point of the set of data points is determined by (a) determining a pressure difference by subtracting a first pressure value associated with a first time from a second pressure value associated with a second time and (b) dividing the pressure difference by a time difference, wherein the time difference comprises a difference between the second time and the first time.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 illustrates an example of a left ventricular pressure tracing displayed by commercially available hemodynamic systems.

FIG. 2 illustrates an example of a ventricular “pressure loop” produced by the disclosed method.

FIG. 3 illustrates a set of pressure loop variants produced by the disclosed method demonstrate characteristics of ventricular blood pressurization.

FIG. 4 illustrates an identification of pre-a wave diastolic pressure by the disclosed method.

FIG. 5 illustrates simultaneous comparison of loop-derived ventricular end-diastolic pressure versus maximum systolic pressure across multiple cardiac cycles (left graph).

FIG. 6 illustrates a comparison of pressure loops produced from the same individual at different time points.

FIG. 7 illustrates an example of a ventricular pressure change per time (dP/dt) versus time plot.

FIG. 8 illustrates an example of standard aortic blood pressure tracing displayed by commercially available hemodynamic systems.

FIG. 9 illustrates an example of an arterial “pressure loop” produced by the disclosed method that is performed for numerous consecutive heart beats.

FIG. 10 illustrates arterial pressure loop variants produced by the disclosed method.

FIG. 11 illustrates simultaneous comparison of arterial pressure loop areas versus arterial pressures.

FIG. 12 illustrates a comparison of pressure loops produced from the same artery at different time points.

FIG. 13 illustrates a determination of diastolic period with minimal cyclic pressure fluctuations.

FIG. 14 illustrates a determination of mono-exponential curve asymptote.

FIG. 15 illustrates a determination of “corrected” Tau.

FIG. 16 illustrates a comparison of calculated versus actual arterial pressures.

FIG. 17 illustrates simultaneous pressure tracings from ventricular and downstream arterial pressure sources.

FIG. 18 illustrates an arterial-to-ventricular pressure ratio produced by the disclosed method during valve stenosis.

FIG. 19 illustrates an arterial-to-ventricular pressure ratio produced by the disclosed method during hypertrophic obstructive cardiomyopathy.

FIG. 20 illustrates arterial-to-ventricular pressure ratio indices produced by the disclosed method.

FIG. 21 illustrates a novel comparison of multiple pressure loops produced by the disclosed method.

FIG. 22 illustrates general features of the disclosed method and interface within a cardiac catheterization laboratory.

FIG. 23 illustrates a typical setup for collection of simultaneous aortic blood pressure (Pa) and distal coronary artery pressure (Pd) during traditional FFR measurements, as well as use of the disclosed method for CFRp measurements (left image).

FIG. 24 illustrates a typical guiding catheter and pressure wire arrangement to simultaneously measure aortic pressure (Pa) and distal pressure (Pd) from the same pressure source (tip of the guide catheter) during pressure “equalization” (left image).

FIG. 25 illustrates simultaneous Pa and Pd measurements during one heartbeat immediately following commercially-available pressure “equalization.”

FIG. 26 illustrates that distal intracoronary pressure measurements (Pd) are reduced compared to the guiding catheter reference measurements (Pa) in the presence of anatomical resistance between the two pressure sources.

FIG. 27 illustrates simultaneous beat-by-beat calculation of mean diastolic 1-Pd/Pa and mean diastolic velocity following an intracoronary bolus of adenosine to induce hyperemia.

FIG. 28 illustrates exemplary beat-by-beat comparison of pressure-derived 1-Pd/Pa versus Doppler-derived blood velocity following adenosine-induced hyperemia and return to baseline coronary flow.

FIG. 29 illustrates exemplary beat-by-beat comparison of diastolic pressure-derived CFR (CFRp) versus diastolic Doppler-based CFR following adenosine-induced hyperemia and return to baseline coronary flow.

FIG. 30 illustrates a flow diagram for a method, in accordance with some embodiments.

FIG. 31 depicts a block diagram illustrating a computing system, in accordance with some embodiments.

### DETAILED DESCRIPTION

Currently-available methods for evaluating characteristics of the heart use time series measurements of blood pressure (ventricular or arterial pressure). These methods may be useful for identifying and treating cardiac abnormalities. Disclosed are systems, methods, and articles of manufacture for producing new blood pressure information to improve detection of heart characteristics, to improve treatment for cardiac abnormalities.

Instead of merely analyzing pressure over time, the disclosed method calculates the time rate of change of pressure (e.g., the time derivative of pressure, or dP/dt), and evaluates this against corresponding time series measurements of pressure. The method produces a representation showing the relationship between dP/dt and corresponding pressure measurements. The representation may use arterial pressure (Pa) or ventricular pressure (Pv). Herein, “ventricular pressure” refers to blood pressure measured within the ventricles of the heart and “arterial pressure” refers to blood pressure measured within the arteries of the heart. For example, the relationship may comprise a set of pairwise relations, with a pairwise relation including a particular Pv value at a particular time and its corresponding dPv/dt. In some cases, this representation is a visual representation. In some cases, the visual representation is a plot (hereinafter referred to as a “pressure loop plot”). The type of pressure used (Pa or Pv) may determine important characteristics of the representation. For example, Pa and Pv may generate different types of pressure loops with different visual features. dPv/dt may be calculated, for example, by calculating a difference in pressure between two samples and dividing by the time difference between the two samples.

The pressure loop plot may have several visual characteristics indicative of heart function. For example, the size of at least a region the loop (or at least a region of the plot), the shape of the loop (or of at least a region of the loop or plot), and aspects of the top, bottom, left, and right sides of the loop may each or in combination provide information relating to proper function or impairment of veins or arteries of the heart.

Pressure loop plots may be analyzed in multiple ways. Many visual features, such as the size, shape, and indentations on the surface of the loops, may be visually inspected. In some cases, the raw dP/dt values and corresponding pressure measurements may be analyzed using or processed by a mathematical model, such as a statistical model or a machine learning model. In some cases, visual plots (such as pressure loops), can be analyzed using a machine learning model, such as a convolutional neural network (CNN).

The pressure data may be collected using invasive or non-invasive cardiac measurement devices. An example of an invasive device is an intracardiac device, such as aa CardioMEMS™ system ir a left ventricular assist device. Examples of non-invasive cardiac measurement devices include ultrasound Doppler, magnetic resonance imaging (MRI), and/or cardiovascular sound intensity devices.

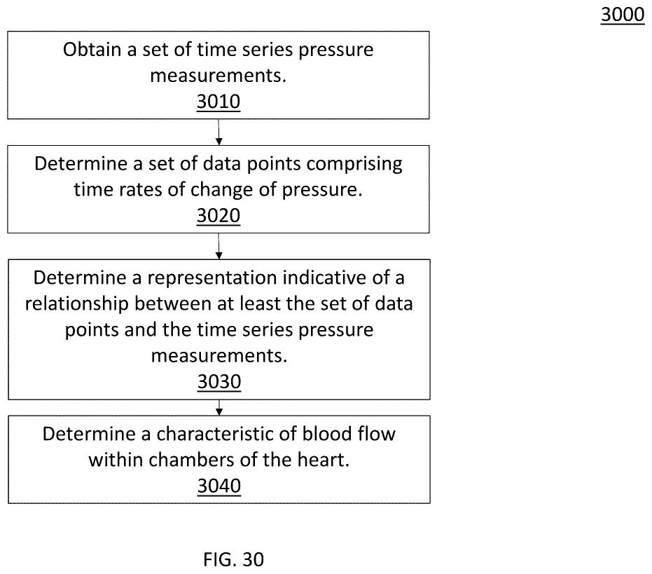

FIG. 30 illustrates a flow diagram for a method 3000, in accordance with some embodiments.

In a first operation 3010, a set of time series pressure measurements is obtained from a subject. The set of time series pressure measurements may be ventricular pressure (Pv) measurements or arterial pressure (Pa) measurements. The time series pressure measurements may be obtained invasively or non-invasively (e.g., without contacting the body of the subject). For example, the measurements may be obtained invasively by an intracardiac device, such as a pulmonary artery hemodynamic monitoring device or a left ventricular support device. The measurements may be obtained non-invasively using a device or apparatus that does not contact the body of the subject and/or is not inserted into the body, such as an ultrasound Doppler device or apparatus, magnetic resonance imaging (MRI) or apparatus, or cardiac sound intensity device or apparatus. In some cases, the time series pressure measurements may be collected at least in part by measuring chamber dimensions and/or ventricular blood pressure. The time series pressure measurements may be collected over a duration comprising one or more heartbeats. In some cases, the duration may be at least one second, at least five seconds, at least 10 seconds, at least 30 seconds, at least one minute, at least ten minutes, at least fifteen minutes, at least half an hour, at least an hour, at least two hours, at least three hours, at least six hours, at least half a day, at least a day, at least a week, at least a month, at least three months, at least six months, or at least one year. In some cases, the duration may be at most five seconds, at most 10 seconds, at most 30 seconds, at most one minute, at most ten minutes, at most fifteen minutes, at most half an hour, at most an hour, at most two hours, at most three hours, at most six hours, at most half a day, at most a day, at most a week, at most a month, at most three months, at most six months, or at most one year. In some cases, the duration may be between one and five seconds, between five and ten seconds, between ten and 30 seconds, between 30 seconds and one minute, between one and ten minutes, between ten and 30 minutes, between 30 minutes and an hour, between one and two hours, between two and three hours, between three and six hours, between six and twelve hours, between twelve hours and one day, between one day and one week, between one week and one month, between one month and two month, between two months and three months, between three months and six months, or between six months and one year.

The subject may be a human subject. In some cases, the subject is a non-human animal. The subject may be a mammalian, avian, reptilian, or amphibian subject. For example, the subject may be a dog, cow, horse, pig, sheep, chicken, turkey, ostrich, mouse, cat, deer, snake, lizard, frog, monkey, ape (e.g., chimpanzee), or another animal.

In a second operation 3020, a set of data points comprising time rates of change of pressure (e.g., ventricular pressure or arterial pressure) is determined from the time series pressure measurements. The time rate of change may be a first derivative of pressure with respect to time (dP/dt), and may relate to ventricular pressure (dPv/dt) or arterial pressure (dPa/dt). The time derivative for a particular pressure value may be calculated by subtracting adjacent pressure measurements in time (e.g., one in the immediate past and one in the immediate future) and dividing them by the time differential between them (e.g., by multiplying by the sampling rate

In a third operation 3030, a representation indicative of a relationship between at least the set of data points and the time series pressure measurements is determined. The relationship may comprise or incorporate a set of pairwise relations between the time rates of change of pressure (dP/dt) values with corresponding pressure values. This representation may comprise raw data, e.g., in a text format, which may be analyzed by a mathematical model. The representation may be pre-processed or compressed. In some cases, a dimensionality reduction method (e.g., an autoencoder) may be used to compress the data in order to generate a feature representation as input to a machine learning algorithm. In some cases, the representation is a visual representation. The visual representation may be a plot. The plot may include one or more loop tracings indicating associations between time rates of change of pressure and pressure values. The loop shape may occur because blood pressure increases and decreases over the course of a heartbeat, resulting in multiple dP/dt values corresponding to a single pressure measurement. The one or more loop tracings may be averaged, and the average may be superimposed on top of the individual loop tracings. The plot comprising the loop tracings is referred to herein as a “pressure loop plot.”

In a fourth operation 3040, a characteristic of blood flow within chambers of the heart is determined by processing the representation. Processing may be performed by inspecting visual features of a pressure loop plot. The processing may be performed using an image processing or computer vision system. The processing may be performed using a mathematical model. Processing may comprise determining associations between visual features of the pressure loop plot and cardiac abnormalities. Characteristics of the pressure loop plot indicative of health may comprise smoothness, a size of the loop, a number of indentations, a size of an indentation, a shape of an indentation, a tangential slope of a point on the plot, a symmetry of the plot, an area of all or a portion of the plot, or a location of a top, bottom, left, or right border of the plot. Characteristics of the heart which may be determined from processing the plot may include ventricular power, ventricular resistance, or ventricular blood flow, elasticity, compliance, contractility stroke volume, or response to a modifying factor.

In some cases, analysis of the representation may be used to determine a course of treatment for a patient with cardiac abnormalities. A treatment may comprise a diet, exercise, a surgery, a course of medication, administration of intravenous (IV) fluid (e.g., saline or lactated ringers), or a combination thereof. In some cases, analysis of the representation is used to screen potential patients. Based on a classification determined from the representation, a patient may be designated as low risk, medium risk, or high risk. Medium or high risk cases may be escalated to appropriate medical personnel.

In some cases, the method may comprise collecting a second set of data points comprising time rates of change of acceleration of pressure (e.g., d³P/dt³). These may be time rates of change of ventricular pressure or arterial pressure. Based at least in part on the second set of data points, the method may evaluate pre a-wave diastolic pressure.

Processing of the pressure measurements may be performed using a computing device. The computing device may be, for example, a laptop computer, desktop computer, tablet computer, smartphone, graphics processing unit (GPU), or personal digital assistant (PDA).

### Ventricular Blood Pressurization Characterization

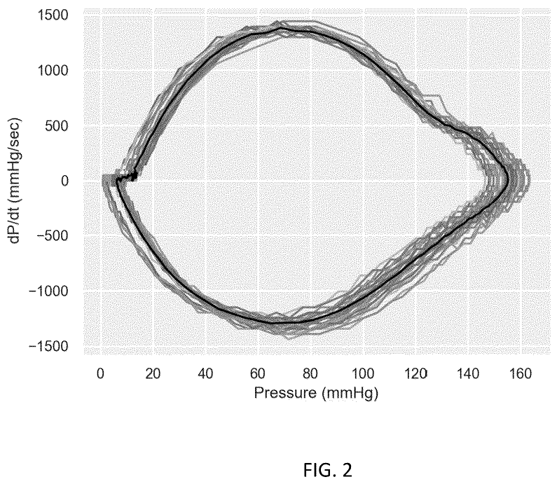

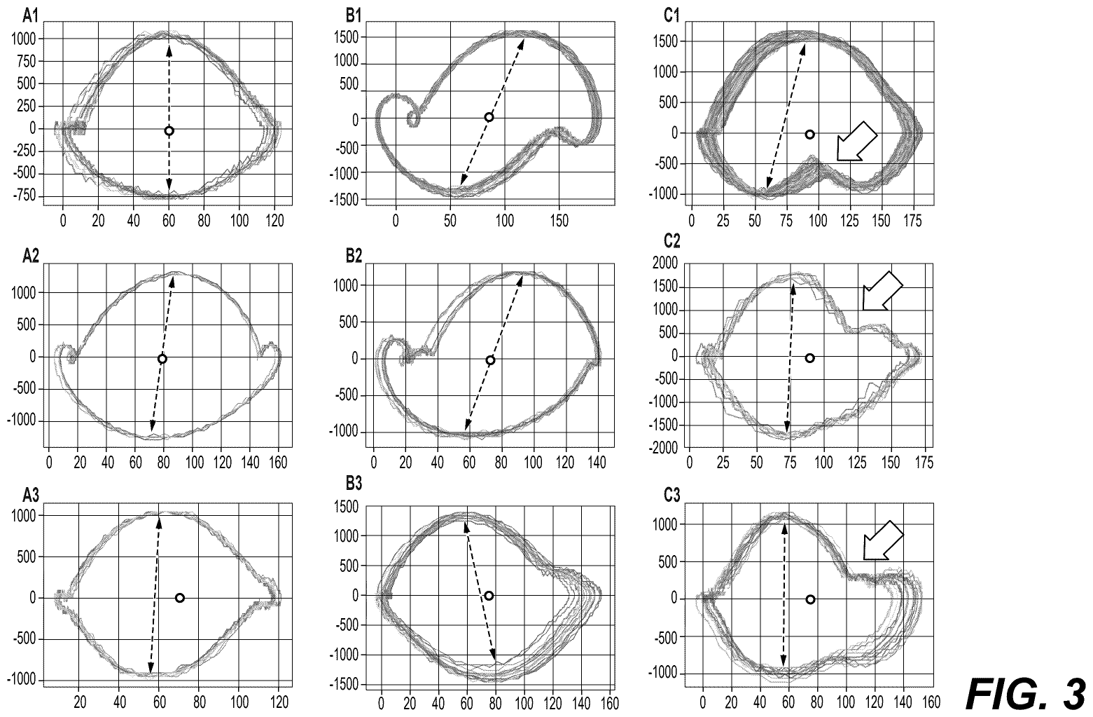

Disclosed herein are systems, methods, and articles of manufacture for performing Pv measurements by transforming the Pv versus time data (often presented using a “time-frequency plot”) into rate of Pv change per change in time (dPv/dt) versus Pv data. In doing so, operators can derive multiple measurements from raw Pv data (either preexisting data or collected real-time) and evaluate ventricular function in new ways. Plotting dPv/dt vs Pv may create a “pressure loop” for each cardiac cycle (FIG. 2) compared to the typical peaks and valleys observable from a time-frequency plot. Standard measurements can be obtained from the ventricular pressure loop, including X-axis minimum (“left border”, minimum diastolic pressure), X-axis maximum (“right border”, maximum systolic pressure), Y-axis maximum (“top border”, maximum systolic dP/dt), and Y-axis minimum (“bottom border”, minimum diastolic dP/dt). Multiple measurements and characteristics can be derived following analysis of the pressure loop (FIG. 3) as discussed below.

Measurements and characteristics derived from analysis of the pressure loop include, but are not limited to including, the following:

Loop size—Total and subtotal areas (e.g., Loop upper half and lower halves, loop quarters, or other-sized portions), axial dimensions, or other measurements associated with an area of or a length or width of at least a portion of a pressure loop. Conceptually, a ventricular “pressure loop” disclosed method may plot dPv/dt versus Pv. The area of the entire loop can be calculated by integration of absolute values within the boundaries of the loop (Equation A1).

$$Pv loop area = {\int }_{Pv start}^{Pv end} \vert f ( Pv ) * dPv \vert \qquad Equation A1$$

(f(Pv) is the instantaneous dPv/dt at any Pv value)

Subareas of the total Pv loop can be calculated by adjusting the limits of integration (e.g., summation of all positive dPv/dt values to calculate area above the X-axis, or summation of all negative dPv/dt values to calculate area below the X-axis). If the raw Pv difference is calculated between two different samples, agnostic of intervening time interval, then multiplication by the sampling rate (e.g., number of samples per second) would then be performed for consistency across different sampling techniques. Also, Pv loop areas, representing ventricular function per cardiac cycle, could be used to calculate ventricular function per time period (Equation A2).

$$Pv loop area / Time = Heart rate * Pv loop area \qquad Equation A2$$

Loop shape—Overall shape characteristics, such as “symmetry (e.g., the degree to which portions of the loop bisected by an axis are similar or identical)”, “smoothness (e.g., a relative lack of indentations or sharp angles or corners)”, presence of abnormal “indentations,” differences between upper and lower half curves/areas, and the tangential slopes for points in different quadrants of the loop, can be determined using the methods described herein. The smoothness of an observed loop segment can be compared to an expected best-fit curved line, then its correlation with the expected curve quantified. A Pv loop shape produced by a ventricular chamber of uncertain characteristics can be compared versus a database of many loop shapes produced by ventricular chambers with known characteristics, then best matches (and known characteristics) reported for the individual loop being tested. Examples of this can include 1) loops of normal size and smoothness resulting from normal ventricles, 2) loops with irregular smoothness resulting from ventricles impaired by coronary artery disease and ischemic cardiomyopathy, 3) loops with regular smoothness and irregular (e.g., smaller) size resulting from ventricles impaired by global non-ischemic cardiomyopathy, and 4) loops with irregular smoothness resulting from ventricles impaired by hypertrophic cardiomyopathy.

Loop cycle duration—The overall number of samples needed to create a complete loop divided by sampling rate can determine the time duration of each loop. Loop duration can then be used to calculate instantaneous heart rate for a single loop, or an average heart rate across multiple loops.

Characteristics of top/bottom borders relative to a reference point—The top border represents the point of maximum rate of blood pressurization (i.e., peak contraction force) while the bottom border represents the point of minimum blood pressurization (i.e., peak relaxation force). These points in the loop may be consistent across multiple pressure loops for a particular subject and can be observed being “aligned”, “rotated”, and/or “shifted” around a reference point (e.g., loop center or X-axis pressure median).

Characteristics of left border—Pressure changes during diastole (making up the left border of the pressure loop) can be characterized (e.g., “drastic” or “subtle”) and help decipher hemodynamically useful timepoints occurring during diastole when Pv can be reported. A method to define the precise moment before atrial contraction (also known as pre-a wave ventricular pressure) is also proposed.

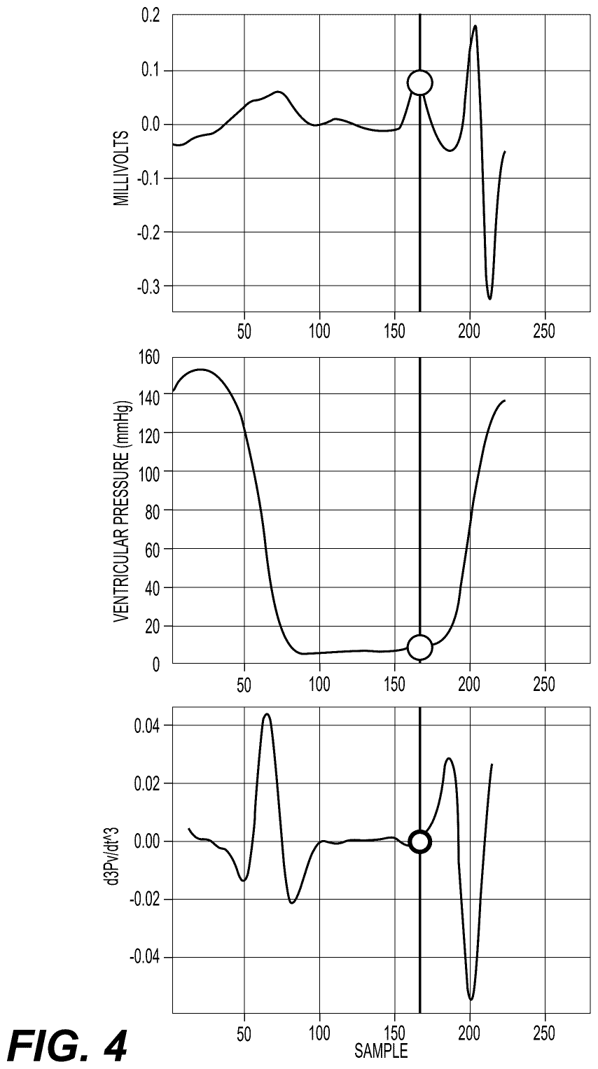

Identification of pre-a wave diastolic pressure—Pre-a wave diastolic pressure measured within a ventricle may be a useful reporting metric because it provides useful information about cardiac function and correlates highly with more direct measurements of upstream atrial pressure (in the absence of intervening valve disease). Thus, if only ventricular pressures are measured, then accurate identification of pre-a wave diastolic pressure provides a useful surrogate of upstream atrial chamber pressure. Identification of the exact moment when to capture this pre-a wave measurement from Pv data can be performed. The disclosed method identifies this moment in a manner by analyzing the third derivative (d³Pv/dt³) of the Pv versus time function (FIG. 4). Analysis of the third derivative of Pv versus time allows the disclosed method to identify the moment before peak systole (or before the dominant Pv upstroke) when (d³Pv/dt³) transitions from negative to positive values. Analogously, if dPv/dt represents the “velocity” of pressure change over time, and second derivative (d²Pv/dt²) represents the corresponding “acceleration”, then the third derivative (d³Pv/dt³) represents the “change in acceleration”. Thus, the disclosed method identifies the precise moment when diastolic (d³Pv/dt³) becomes consistently positive, which represents the moment when ventricular pressure concludes peak “deceleration” towards lower values and begins positive “change in acceleration” towards higher values. Within the heart, this is the moment cardiac muscle begins to contract within the atrium followed by muscle contraction within the ventricle.

Characteristics of right border—Pressure changes during maximum ventricular systolic pressure (making up the right border of the pressure loop) may reflect the effect of pressure wave reflections and can be characterized or quantified. It also shows variation of max Pv across multiple loops.

Timing of loop features—The occurrence (or timing) of distinct loop features such as left/right/top/bottom borders can be recorded and used to identify the occurrence of systole, diastole, or other periods of interest.

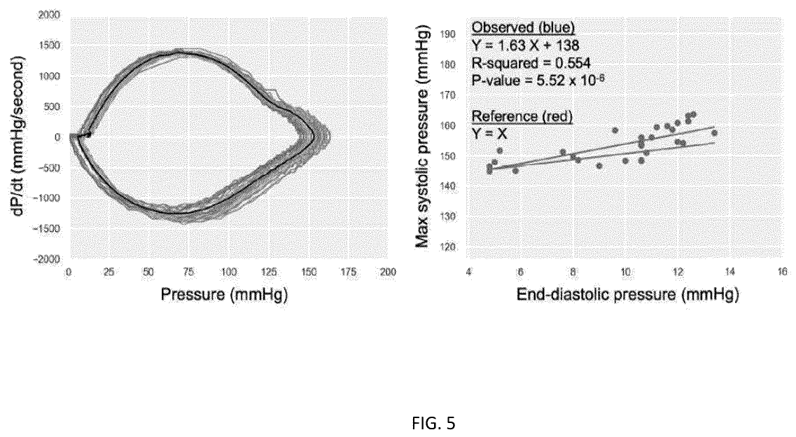

Simultaneous comparison of various parameters—Simultaneous comparison of any loop-derived factors could be performed. An example is comparison of ventricular end-diastolic pressure versus maximum Pv across multiple beats. This comparison reveals a relationship (FIG. 5) that appears similar to, but is fundamentally different from, the classic Frank-Starling curve that reflects the relationship between ventricular end-diastolic volume and stroke volume. Another example is end-diastolic pressure versus loop size. Another example is loop size versus trans-valvular pressure gradients or trans-valvular pressure ratios.

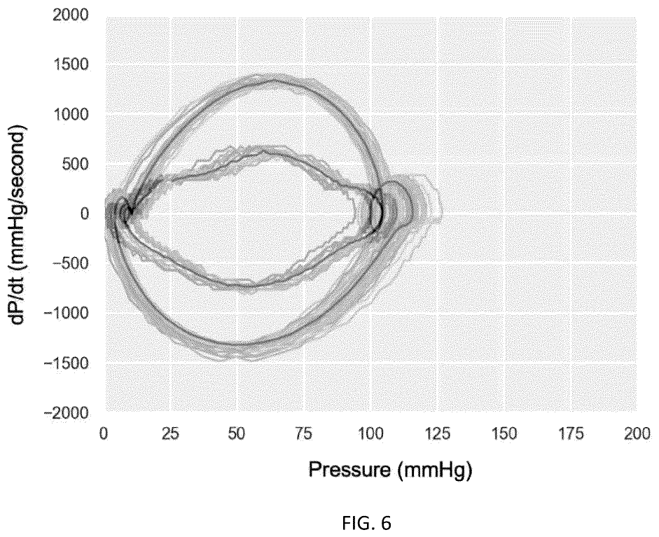

Comparison of parameters between different time points or different individuals—Various pressure loop measurements can be compared over across different time points, an example being total loop area before and after a heart attack (myocardial infarction) to demonstrate worsening of ventricular function, or before and after heart transplantation to demonstrate improvement of ventricular function (FIG. 6). Efficacy of cardiac therapies, particularly procedures and pharmacologic agents, could be tested in this way. Loop measurements can also be compared between different individuals/populations to identify objective differences in cardiac function.

Downstream calculations using ventricular pressure loop data—Primary data within the ventricular pressure loop can be used to calculate unique secondary measurements. An important application example of this function is how ventricular pressure loop data can be used to calculate ventricular “power”, “resistance”, and “flow” measurements. Power calculations of the left ventricle may calculate downstream flow and resistance within the systemic circulation of the body, while power calculations of the right ventricle may calculate downstream flow and resistance within the pulmonary circulation of the lung.

Ventricular power: In creating ventricular pressure loops, the disclosed method allows a comparison of dPv/dt (mmHg/sec units) versus Pv (mmHg units) at any point in time, rather than a comparison of dPv/dt versus time itself. Within the disclosed method's comparison, raw blood pressure is mathematically equivalent to work performed (or energy stored) in Joules per volume of blood (Equation A3).

$$\frac{1 mm Hg}{133.322387415} = Pascal = \frac{kg}{m * {sec}^{2}} = \frac{Newton}{{m}^{2}} = \frac{Joule}{{m}^{3}} \qquad Equation A3$$

Blood pressure per time is mathematically equivalent to power (or energy stored per unit time) in Watts per volume of blood (Equation A4).

$$\frac{1 mm Hg / sec}{133.322387415} = \frac{Newton / sec}{{m}^{2}} = \frac{Joule / sec}{{m}^{3}} = \frac{Watt}{{m}^{3}} \qquad Equation A4$$

Thus, the disclosed method (as well as the corresponding system and article or manufacture) creates unique pressure loops that allow analyses of ventricular power that is indexed by ventricular blood volume.

The quantification of ventricular power index in this manner can be analyzed for different parts of the cardiac cycle (i.e., systole alone, diastole alone, the entire cardiac cycle). Conceptually, the ventricular power analysis also performs pressure-weighted averaging of the power index across different Pv values, as opposed to native time-weighted averaging of the power index across different time values (Equation A5 and FIG. 6).

$$Power index Mean = \frac{1}{T start - T end} {\int }_{T start}^{T end} \vert f ( T ) * dT \vert \qquad Equation A5$$

(f(T) is the instantaneous dPv/dt at any T value of time)

In doing so, the disclosed system, method, and article of manufacture may emphasize power index values produced during peak systole/diastole that 1) are quickly generated within a relatively short time period and are underemphasized within time-weighted data, and 2) may more closely quantify ventricular function.

Moreover, the disclosed subject matter may indicate (e.g., report and the like) ventricular power in multiple different ways including, but not limited to, the measurements below:
Sum of pressure-weighted power index values across different parts of the cardiac cycle, or simply the pressure loop area (adapted from Equation A1).

$$Power index Sum = {\int }_{P start}^{P end} \vert f ( Pv ) * dPv \vert \qquad Equation A1 modified$$

Maximum of pressure-weighted power index values during systole (maximum dP/dt value).
Minimum of pressure-weighted power index values during diastole (minimum dP/dt value).
Mean of pressure-weighted power index values across different parts of the cardiac cycle (Equation A6).

$$Power index Mean = \frac{1}{{\int }_{P start}^{P end} \vert dPv \vert} {\int }_{P start}^{P end} \vert f ( Pv ) * dPv \vert \qquad Equation A6$$

Root mean square (RMS) of pressure-weighted power index values across different parts of the cardiac cycle (Equation A7).

$$Power {index}_{RMS} = \sqrt{\frac{1}{{\int }_{P start}^{P end} \vert dPv \vert} {\int }_{P start}^{P end} \vert {f ( Pv )}^{2} * dPv \vert} \qquad Equation A7$$

Downstream blood flow and resistance: Following the disclosed method's calculation of ventricular power indexed by blood volume, the disclosed method can create estimates of downstream blood flow and resistance by using existing equations of electrical power, resistance, and flow (Equation A8).

$$Resistance = \frac{{Voltage}^{2}}{Power} = \frac{Power}{{Flow}^{2}} = \frac{Voltage}{Flow} \qquad Equation A8$$

While such equations use actual power values, and not power indexed by volume as produced by the disclosed method, a downstream resistance “factor” calculation by the disclosed method would be a creation (Equation A9) with the units mmHg-see or dynes-sec/cm².

$$Resistance factor = \frac{Mean arterial {pressure}^{2}}{Ventricular power index} \qquad Equation A9$$

Calculation of actual resistance could be achieved by including blood volume into the equation (Equation A10) with the units mmHg-sec/m or dynes—sec/cms.

$$Resistance = \frac{Mean arterial {pressure}^{2}}{Ventricular power index * Blood volume} \qquad Equation A10$$

In the same way, a downstream cardiac output or blood flow “factor” calculation by the disclosed method would be a creation (Equation A11) with the unit sec⁻¹.

$$Blood flow factor = \frac{Mean arterial pressure}{Resistance factor} \qquad Equation A11$$

Calculations of actual blood flow could be achieved by including blood volume into the equation (Equation A12) with the units m³/sec.

$$Blood flow = \frac{Mean arterial pressure}{( \frac{Resistance factor}{Blood volume} )} \qquad Equation A12$$

The disclosed subject matter may also have the ability to transform non-invasive ventricular pressure waveforms into pressure loops. Examples of this include non-invasive ultrasound Doppler, magnetic resonance imaging, and cardiovascular sound intensity to measure the velocity of ventricular blood flow for one or more cardiac cycles and calculate corresponding ventricular blood pressure. The derived pressure waveforms from each of these various techniques could be transformed into pressure loops and analyzed as described above.

Hypothetically, subtle variations of a pressure loop can be created, such as using change in Pv per sample (dPv/sample) rather than dPv/dt. Different units of pressure or time could also be used. These variations would effectively change the scale of the pressure loops demonstrated here and any established ranges for “normal” measurements would also change, but the underlying concepts and principles of application would be identical.

The disclosed subject matter and its Pv pressure loops could be used by existing commercial devices, such as those that can acquire Pv measurements from a ventricle, to improve baseline function. Examples include pulmonary artery hemodynamic monitoring device (e.g., Swan-Gantz catheter) inserted through a central vein, right atrium, right ventricle, and pulmonary artery; right ventricular pressure loops could be monitored and analyzed using pressure loops. Left ventricular support devices (e.g., Impella mechanical pumps) monitor aortic pressure and estimate left ventricular pressure. Modifying the Impella device to directly measure Pv, or using calculated Pv, could be used to create Pv loops to monitor left ventricular function and determine appropriateness for escalation/de-escalation of cardiac support.

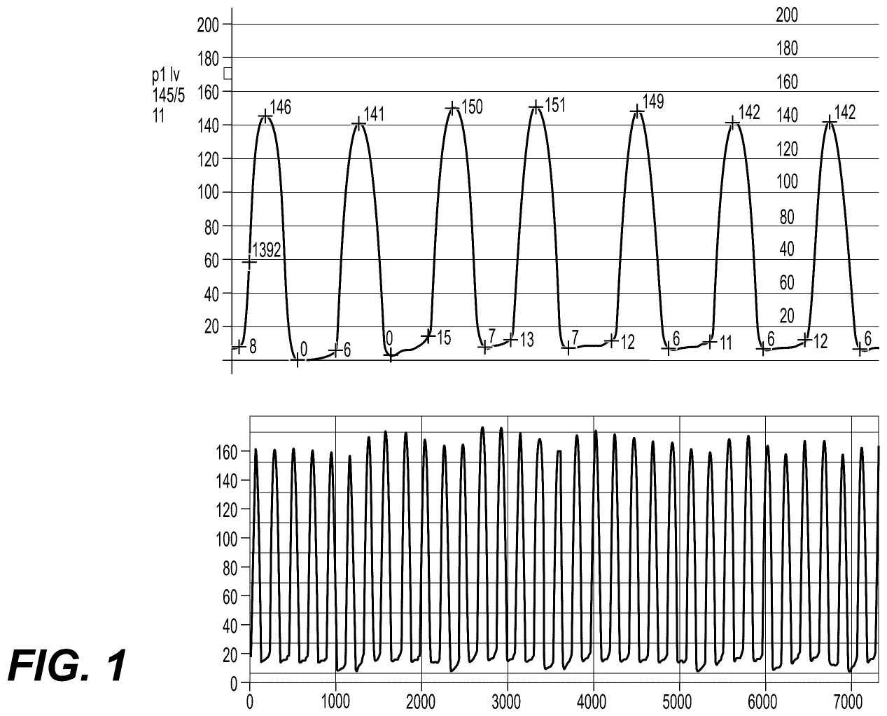

FIG. 1 illustrates an example of a left ventricular pressure tracing displayed by commercially available hemodynamic systems. Measurements from the first cardiac beat include maximum systolic pressure of 146 mmHg, minimum diastolic pressure 0 mmHg, end diastolic pressure 6 mmHg, and maximum

$$\frac{dP}{dt}$$

of 1392 mmHg/sec.

FIG. 2 illustrates an example of a ventricular “pressure loop” produced by the disclosed method. A pressure loop determination is performed for each of numerous consecutive heart beats. The determined pressure loops are then overlaid upon each other (using the same raw data source as FIG. 1). The black line illustrates an average (e.g., a mean dP/dt for each Pv value) of all pressure loop curves. The left and right borders indicate points of minimum diastolic pressure and maximum systolic pressure, respectively. The top border and bottom border amplitudes indicate maximum systolic dP/dt and minimum diastolic dP/dt, respectively.

FIG. 3 illustrates a set of pressure loop variants produced by the disclosed method demonstrate characteristics of ventricular blood pressurization. “Smooth” and “symmetric” loops are demonstrated in column A compared to “slanting” (shown by dashed double arrow connecting max and min dP/dt points) in column B and loop “indentation” (solid arrows) in column C. The point locations of max/min dP/dt appear to “rotate” around the pressure median (small circles) in some panels (A1-2 and B1-2) but are “shifted” off the pressure median others (row 3 and column C). While areas contained within the upper and lower curves of each loop (above/below the X-axis) appear to be equal in most panels, the upper curve can appear relatively larger (panels A1 and C1) or smaller (panel C3) than the corresponding lower curve. Pressure changes during diastole (points along left border) can be drastic (panels B1-2) or subtle (row 3 and column 3). Units for each graph are mmHg/second (Y-axis) and mmHg (X-axis).

FIG. 4 illustrates an identification of pre-a wave diastolic pressure by the disclosed method. The disclosed method identifies the precise moment when the third derivative of the ventricular pressure versus time function is consistently positive before peak systole occurs. This moment represents when ventricular pressure concludes peak “deceleration” towards lower values and begins positive “change in acceleration” towards higher values. Ventricular pressure at this moment corresponds with pre-a wave pressure, which provides useful information about cardiac function and correlates highly with direct measurements of upstream atrial pressure (in the absence of intervening valve disease). FIG. 4 shows an example of an electrocardiogram tracing (top panel), left ventricular pressure tracing (middle panel), and third derivative d³Pv/dt³ tracing (bottom panel) acquired simultaneously from the same source. The precise moment when d³Pv/dt³ becomes consistently positive before peak systole is identified by the disclosed method (circle at sample number 166), with corresponding normal left ventricular pressure of 9.2 mmHg and electrocardiogram timing on the P-wave shown (circles).

FIG. 5 illustrates simultaneous comparison of loop-derived ventricular end-diastolic pressure (EDP: points along left border) versus maximum systolic pressure (Pv: points along right border) across multiple cardiac cycles (left graph). The resulting relationship between maximum Pv and EDP is linear (right graph) and in this example has a slope greater than 1 (red reference line). This suggests that for every 1 mmHg increase in EDP achieved, there is a >1 mmHg relative increase in maximum Pv.

FIG. 6 illustrates a comparison of pressure loops produced from the same individual at different time points. The smaller inner loop is related to abnormal ventricular function, while the larger outer loop is related to normal ventricular function.

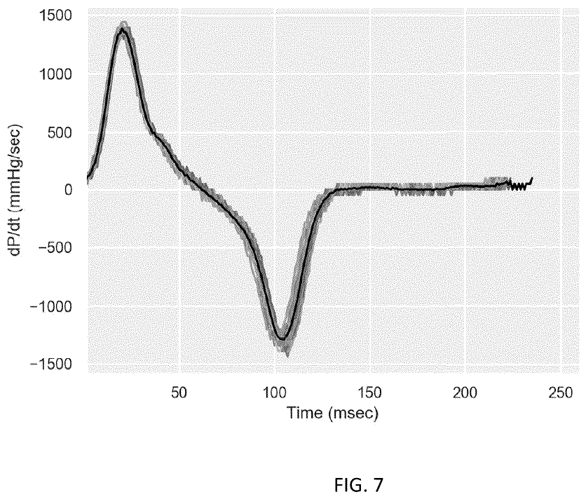

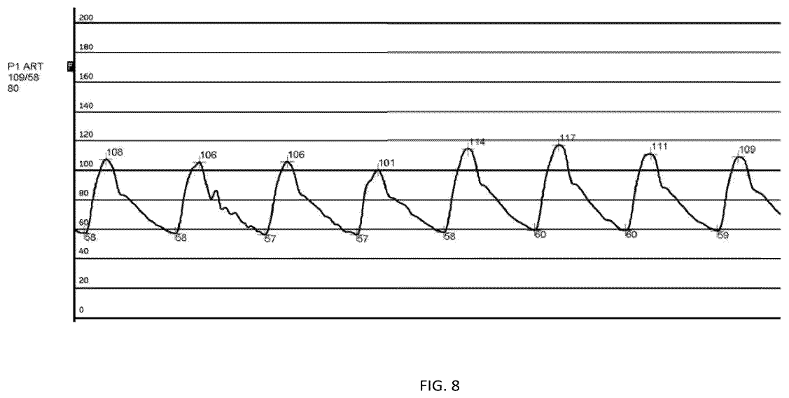

FIG. 7 illustrates an example of a ventricular pressure change per time (dP/dt) versus time plot. Positive dP/dt values occur during peak systole, while negative dP/dt values occur during peak diastole. In this time-dependent plot, the time period between the diastolic peak and next systolic peak (occurring from about 130 msec to 230 msec), which represents ventricular passive relaxation, has greater weight than compared to its graphical representation within the disclosed method's corresponding pressure loop (FIG. 2 left border). In comparison, the disclosed method's pressure loop (plotting dP/dt versus pressure) weighs ventricular dP/dt values evenly across a range of ventricular pressures rather than across a range of sampling times. In doing so, peak ventricular systole/diastole are emphasized, while end systole/diastole are deemphasized, within the disclosed method's pressure loop analyses.

### Arterial Blood Pressurization

Arterial blood pressure (Pa) and heart rate are ubiquitous vital signs used worldwide to inform clinicians about patients' cardiovascular health. Elevated blood pressure defines hypertension and reduced blood pressure defines hypotension. An accentuated separation between systolic blood pressure (SBP: identified by maximum blood pressure) and diastolic blood pressure (DBP: identified by minimum pressure) can indicate additional cardiovascular abnormalities. Both an elevated and a reduced heart rate can result from a primary electrical conduction disturbance of the heart or as a secondary response to a different cardiovascular abnormality. Critically reduced blood pressure or heart rate values further from normal values and closer to zero can signify cardiac death.

Invasive measurement of Pa is routinely preformed in patients undergoing invasive evaluation within a hospital's cardiac catheterization laboratory. Pa can also be measured continuously for those being monitored in operating rooms, emergency rooms, or intensive care units. Pa measurement is particularly useful to evaluate those suffering from “shock” states (where the amount of blood flow provided from the heart to the body is insufficient to meet the body's metabolic demands). Continuous Pa recordings produce classic pressure versus time waveforms (see, e.g., FIG. 1) that result from sequential heart beats and form the basis of routine measurements such SBP, DBP, and mean arterial pressure. The raw information contained within continuous Pa waveforms also provide blood pressure and heart rate vital signs that are used worldwide. Pa measurements are also integrated into existing secondary measures of cardiac output (CO) and resistance (R) to blood flow (Equations B1-5). More complex systolic Pa integration (Equation B6) or Pa averaging (Equation B7-10) techniques have been used for various applications including estimation of CO and quantification of cardiac valve stenosis.

Despite the broad usefulness of Pa waveforms for vital sign measurement and more advanced secondary calculations, the available tools to assist clinicians in identifying and quantifying subtle changes in Pa waveform characteristics is limited. Furthermore, unrecognized Pa waveform characteristics may provide new vital signs for clinicians to use worldwide. This section describes Pa waveform analysis performed by the disclosed method to produce and measure cardiovascular vital signs.

### Relevant Equations:

### Analogs of Ohm's Law:

$$Cardiac Output = \frac{Pressure gradient}{Resistance} \qquad Equation B1$$

$$Systemic circulation output = \frac{( MAP - CVP )}{Systemic vascular resistance} \qquad Equation B2$$

$$Pulmonary circulation output = \frac{( mPAP - LAP )}{Pulmonary vascular resistance} \qquad Equation B3$$

(MAP=mean arterial pressure; CVP=central venous pressure; mPAP=mean pulmonary artery pressure; LAP=left atrial pressure)

### Calculation of Resistance (WU):

$$Resistance = \frac{Pressure gradient}{Cardiac output} \qquad Equation B4$$

### Cardiac Power Output (Watts):

$$Cardiac power output = \frac{MAP \times Cardiac output}{451} \qquad Equation B5$$

### Pulse Contour Cardiac Output (PCCO):

$$PCCO = Cal \times HR \times \int ( \frac{P ( t )}{SVR} + C ( p ) \times \frac{dP}{dt} ) dt \qquad Equation B6$$

(Cal=patient specific calibration factor; HR=heart rate; P(t)=pressure under the pressure curve; SVR=systemic vascular resistance; C(p)=compliance;
dP/dt=shape of pressure curve)

### Gorlin Valve Area Calculation:

$$AVA = \frac{Cardiac output}{SEP \times HR \times 44.3 \times \sqrt{mean pressure gradient}} \qquad Equation B7$$

$$MVA = \frac{Cardiac output \times 0.85}{DEP \times HR \times 44.3 \times \sqrt{mean pressure gradient}} \qquad Equation B8$$

(AVA=aortic valve area; SEP=systolic ejection period; HR=heart rate; mean gradient across stenotic valve during antegrade flow; MVA=mitral valve area; DEP=diastolic ejection period)

### Hakki Valve Area Estimation:

$$AVA = \frac{Cardiac output}{\sqrt{mean aortic valve pressure gradient}} \qquad Equation B9$$

$$MVA = \frac{Cardiac output}{\sqrt{mean mitral valve pressure gradient}} \qquad Equation B10$$

Pressure loops using either invasive or non-invasive Pa measurements are created by transforming traditional Pa versus time data (also known as a “time-frequency plot”) into rate of Pa change (dPa/dt) versus Pa data. In doing so, operators can derive multiple measurements from raw Pa measurements (which may be either preexisting data or collected in real time) and evaluate the characteristics of arterial pressurization in new ways. Plotting dPa/dt vs Pa creates a “pressure loop” for each cardiac cycle (FIG. 2) compared to the typical peaks and valleys seen with the time-frequency plot. Standard measurements can be obtained from the Pa pressure loop, including X-axis minimum (“left border”, minimum diastolic pressure), X-axis maximum (“right border”, maximum systolic pressure), Y-axis maximum (“top border”, maximum dPa/dt), and Y-axis minimum (“bottom border”, minimum dPa/dt). Novel measurements and characteristics derived from analysis of the pressure loop (FIG. 3) include, but are not limited to those listed below. Also, each of these characteristics derived from arterial blood pressure data may developed into a possible cardiovascular “vital sign” for future use. In fact, the characteristic #3 forms the basis for heart rate, while characteristics #5-6 form the basis for blood pressure.

Loop size—Total and subtotal areas (e.g., Loop upper half and lower halves, loop quarters), axial dimensions, or other quantities associated with an area or perimeter of the loop. An arterial “pressure loop” may be created by the disclosed method by plotting dPa/dt versus Pa. The area of the entire loop can be generally calculated by integration of absolute values within the boundaries of the loop (Equation B11).

$$Pa loop area = {\int }_{P start}^{P end} \vert f ( Pa ) * d Pa \vert \qquad Equation B11$$

(f(Pa) is the instantaneous dPa/dt at any Pa value)

Subareas of the total Pa loop can be calculated by adjusting the limits of integration (e.g., summation of all positive dPa/dt values to calculate area above the X-axis, or summation of all negative dPa/dt values to calculate area below the X-axis).

Additional adjustments to the limits of integration can be introduced in the context of loops cyclic pressure fluctuations (FIG. 3). If the raw Pa difference is calculated between two different data samples, agnostic of intervening time interval, then multiplication by the sampling rate (samples per time) would then be performed for consistency across techniques. Also, Pa loop areas, representing arterial function per cardiac cycle, could be used to calculate arterial function per time period (Equation B12).

$$Pa loop area / Time = Heartrate \times Pa loop area \qquad Equation B12$$

Loop shape—Overall shape “symmetry”, “smoothness”, presence of abnormal “indentations”, differences between upper and lower half curves/areas. The range of slopes for different quadrants of the loop could also be determined. The smoothness of an observed loop segment can be compared to an expected best-fit curved line, then quantified for correlation with the expected curve. An individual loop shape derived from a source with uncertain characteristics can be compared versus a database of many loop shapes derived from sources with known characteristics, then best matches (and associated source characteristics) reported for the individual loop. Examples of this can include 1) loops with irregular smoothness indirectly resulting from ventricles impaired by coronary artery disease and ischemic cardiomyopathy, 2) loops with regular smoothness indirectly resulting from ventricles impaired by global non-ischemic cardiomyopathy, and 3) loops with irregular smoothness indirectly resulting from ventricles impaired by hypertrophic cardiomyopathy.

Loop cycle duration—Overall number of samples needed to create a complete loop that can be divided by sampling rate to determine the time duration of each loop. Loop duration can then be used to calculate instantaneous heart rate for a single loop, or an average heart rate across multiple loops.

Characteristics of top/bottom borders relative to a reference point—The top border represents the point of maximum rate of blood pressurization (i.e., peak contraction force) while the bottom border represents the point of minimum blood pressurization (i.e., peak relaxation force). These points in the loop are consistent across multiple pressure loops and can be observed being “aligned”, “rotated”, and/or “shifted” around a reference point (e.g., loop center or X-axis pressure median).

Characteristics of left border—Pressure changes during diastole (making up the lower left quadrant of the pressure loop) can be characterized (e.g., “flat” or “stable”) and help decipher the timing of diastole without cyclic pressure fluctuations. This diastolic timing can help with additional calculations described in this supplement.

Characteristics of right border—Pressure changes during maximum arterial systolic pressure (making up the right border of the pressure loop) may reflect the effect of pressure wave reflections and can be characterized/quantified. These may also show variation of max Pa across multiple loops.

Characteristics of cyclic pressure fluctuations—Each pressure loop can demonstrate repetitive dPa/dt “indentations” or fluctuations that can be identified and characterized (e.g., incidence number, timing, frequency, or amplitude).

Timing of loop features—The occurrence (or timing) of distinct loop features such as left/right/top/bottom borders can be recorded and used to identify the occurrence of systole, diastole, or other periods of interest.

Simultaneous comparison of various parameters—Simultaneous comparison of any loop-derived factors could be performed. Examples include comparisons of arterial loop size versus minimum/maximum Pa across multiple heartbeats, which reveal a linear relationship (FIG. 4).

Comparison of parameters between different time points or different individuals—Various pressure loop measurements can be compared over across different time points, an example being total loop area before versus after a heart attack (myocardial infarction) to demonstrate worsened cardiac function, or before versus after heart transplantation to demonstrate improved cardiac function (FIG. 5). This type of comparison is similar to, but distinct from, the comparison of ventricular pressure loops (FIG. 5). Efficacy of cardiac therapies, particularly procedures and pharmacologic agents, could be tested in this way. Loop measurements can also be compared between different individuals/populations to identify objective differences in cardiac function.

Downstream calculations using arterial pressure loop data—Primary data within the arterial pressure loop can be used to calculate unique secondary measurements. An important application example of this function is how arterial pressure loop data can be used to estimate ventricular pressure loop data and its secondary measurements.

Estimation of ventricular pressure loop area: Features of arterial pressure loops can be used to estimate the values and features of ventricular pressure loops. In particular, the portion of the arterial pressure loop occurring during systole can be used to estimate the corresponding portion of the ventricular pressure loop occurring during systole (portion of arterial and ventricular pressure loops graphed above the X-axis). Both upper loops resemble oval shapes with the following area calculation (Equations B13 and B14):

$$Pv upper loop area \approx \frac{π}{2} * height * width = \frac{π}{2} * [ max \frac{dPv}{dt} ] [ \frac{1}{2} ( max Pv - min Pv ) ] \qquad Equation B13$$

$$Pa upper loop area \approx \frac{π}{2} * height * width = \frac{π}{2} * [ max \frac{d Pa}{dt} ] [ \frac{1}{2} ( max Pa - min Pa ) ] \qquad Equation B14$$

Pa upper loop area can be used to estimate Pv upper loop area by scaling the height and width of the Pa upper loop to match parameters of the Pv upper loop (Equation B15).

$$\frac{π}{2} * [ max \frac{dPv}{dt} ] [ \frac{1}{2} ( max Pv - min Pv ) ] \approx \frac{π}{2} * s [ max \frac{d Pa}{dt} ] * s [ \frac{1}{2} ( max Pa - min Pa ) ] \qquad Equation B15$$

Where scaling factor “s” equals the width of the ventricular pressure loop (maximum Pv−minimum Pv) divided by the width of the arterial pressure loop (maximum Pa−minimum Pa):

$$Pv upper loop area \approx \frac{π}{2} * {[ \frac{max Pv - min Pv}{max Pa - min Pa} ]}^{2} ( max \frac{d Pa}{dt} ) ( max Pa - min Pa )$$

This equation simplifies to equation B16:

$$Pv upper loop area \approx \frac{π}{2} * \frac{{( max Pv - min Pv )}^{2}}{( max Pa - min Pa )} ( max \frac{d Pa}{dt} ) \qquad Equation B16$$

Assuming that Pv upper loop area is roughly equivalent to Pv lower loop area, then Pv total loop area can be estimated using Equation B17:

$$Pv total loop area \approx π * \frac{{( max Pv - min Pv )}^{2}}{( max Pa - min Pa )} ( max \frac{d Pa}{dt} ) \qquad Equation B17$$

Unknown maximum Pv could be estimated using maximum Pa (assuming there is no pressure gradient from the ventricle to downstream artery). Unknown minimum Pv could be estimated using pulmonary capillary wedge pressure, left atrial pressure, or closest approximation.

Estimation of individual ventricular dP/dt and Pv values: Estimating ventricular Pv and dP/dt values can facilitate the calculation of other ventricular loop downstream measurements. Pv values can be estimated from Pa values using Equation B18:

$$estimated Pv \approx \frac{( max Pv - min Pv )}{( max Pa - min Pa )} * ( Pa - min Pa ) + min ( Pv ) \qquad Equation B18$$

Likewise, ventricular dPv/dt values can be estimated from arterial dPa/dt values using Equation B19:

$$estimated dPv / dt \approx \frac{( max Pv - min Pv )}{( max Pa - min Pa )} * f ( Pa ) \qquad Equation B19$$

(f(Pa) is the instantaneous dPa/dt at any Pa value)

Maximum dPv/dt value can be estimated from maximum arterial dPa/dt value using Equation B20:

$$max dPv / dt \approx \frac{( max Pv - min Pv )}{( max Pa - min Pa )} * max f ( Pa ) \qquad Equation B20$$

Estimation of ventricular power: Following the disclosed method's estimation of Pv and dPv/dt values from arterial pressure loop data, ventricular power can then be estimated using Equations A1, A6, and A7 reproduced below:

$$\begin{matrix}Equation A1 modified & \\ Power index sum = {\int }_{P start}^{P end} \vert f ( Pv ) * dPv \vert & \end{matrix}$$

$$Power index Mean = \frac{1}{{\int }_{P start}^{P end} \vert dPv \vert} {\int }_{P start}^{P end} \vert f ( Pv ) * dPv \vert \qquad Equation A6$$

$$Power index RMS = \sqrt{\frac{1}{{\int }_{P start}^{P end} \vert dPv \vert} {\int }_{P start}^{P end} \vert {f ( Pv )}^{2} * dPv \vert} \qquad Equation A7$$

Estimation of downstream blood flow and resistance: Following the disclosed method's estimation of ventricular power from arterial pressure loop data, measurements of downstream blood flow and resistance can be calculated using Equations A9, A10, A11, and A12 reproduced below:

$$Resistance factor = \frac{Mean arterial {pressure}^{2}}{Ventricular power index} \qquad Equation A9$$

$$Resistance = \frac{Mean arterial {pressure}^{2}}{Ventricular power index * Blood volume} \qquad Equation A10$$

$$Blood flow factor = \frac{Mean arterial pressure}{Resistance factor} \qquad Equation A11$$

$$Blood flow = \frac{Mean arterial pressure}{( \frac{Resistance factor}{Blood volume} )} \qquad Equation A12$$

Calculation of arterial power, blood flow, and resistance: While arterial pressure loops can be used to estimate ventricular pressure loop data and its downstream power, flow, and resistance calculations, the same measurements can also be performed directly from the arterial pressure loops, shown in Equations B21 through B27. Discrepancies between ventricular and arterial power, blood flow, and resistance need to be studied in real life scenarios and may differ in terms of hypothetical limits of power, flow, and resistance (related to ventricular function) versus observed power, flow, and resistance (related to arterial function).

$$Arterial Power index - Sum = {\int }_{P start}^{P end} \vert f ( P a ) * dPa \vert \qquad Equation B21$$

$$Arterial Power index - Mean = \frac{1}{{\int }_{P start}^{P end} \vert dPa \vert} {\int }_{P start}^{P end} \vert f ( P a ) * dPa \vert \qquad Equation B22$$

$$Arterial power index - RMS = \sqrt{\frac{1}{{\int }_{P start}^{P end} \vert dPa \vert} {\int }_{P start}^{P end} \vert {f ( P a )}^{2} * dPa \vert} \qquad Equation B23$$

$$Arterial resistance factor = \frac{Mean arterial {pressure}^{2}}{Arterial power index} \qquad Equation B24$$

$$Arterial resistance = \frac{Mean arterial {pressure}^{2}}{Arterial power index * Blood volume} \qquad Equation B25$$

$$Arterial blood flow factor = \frac{Mean arterial pressure}{Arterial Resistance factor} \qquad Equation B26$$

$$Arterial blood flow = \frac{Mean arterial pressure}{( \frac{Arterial Resistance factor}{Blood volume} )} \qquad Equation B27$$

The disclosed subject matter is not dependent on invasive Pa measurements to perform its analysis. It also has the ability to transform non-invasive arterial pressure waveforms into pressure loops. Examples of this could include non-invasive ultrasound Doppler and magnetic resonance imaging to measure the velocity of arterial blood for one or more cardiac cycles and calculate corresponding arterial blood pressure. The derived pressure waveform could be transformed into pressure loops and analyzed as described. Vibrations and sound produced from blood traveling through arteries, and derivation of an arterial blood pressure waveform, could also be transformed into an arterial pressure loop and analyzed as described above. It is also conceivable that noninvasive blood pressure cuff measurements could be manipulated to produce an arterial blood pressure loop and analyzed as described above. Whatever the means of producing an arterial blood pressure waveform and/or an arterial pressure loop as described above, the disclosed subject matter's 1) creation of the arterial pressure loop and 2) analysis as described, above are both targets for patent protection.

When patients are evaluated for medical care worldwide, vital signs are measured from arterial blood pressure versus time data. Ubiquitous vital signs include arterial blood pressure and heart rate. As described above, the disclosed subject matter further transforms arterial blood pressure versus time data into unique dPa/dt vs Pa pressure loops that can be used to not only characterize how arterial blood is pressurized, but also estimate measurements of ventricular blood pressurization. Each of these unique arterial and ventricular blood pressure measurements performed by the disclosed subject matter can be used as vital signs to identify disease states in patients.

Similar to blood pressure and heart rate metrics, it is foreseen that abnormally sized or shaped arterial pressure loops may indicate an abnormal cardiovascular state. Like other vital signs, significantly reduced pressure loop size below normal and closer to zero may also signify cardiac death. It is also anticipated that the disclosed subject matter will be able to create pressure loops and calculate pressure loop area vital signs from either invasive or non-invasive measurement of blood pressure, such as from an indwelling arterial pressure line or non-invasive blood pressure cuff, respectively.

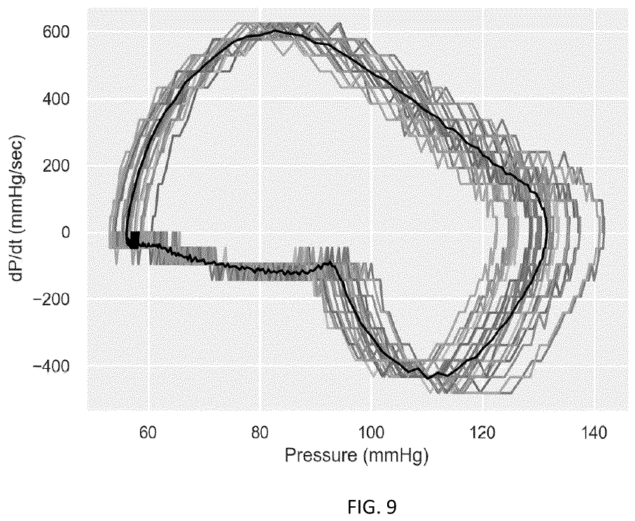

Disclosed herein is an arterial pressure loop graphically representing instantaneous pressure change per unit time (y=dPa/dt) versus arterial pressure (x=Pa)(FIG. 9). Generally speaking, this function converts the pressure versus time waveform generated from each heartbeat into a uniquely shaped loop that can be analyzed alone, compared against other beats from the same patient, and/or compared against other beats from different patients. The size, shape, symmetry, and position of each graphed loop facilitate multiple unique analyses. Measurement examples include left, right, top and bottom points along this loop that correlate to DBP, SBP, maximum dPa/dt, and minimum dPa/dt, respectively. The average center of the loop represents both MAP and pressure change over the entire beat. Loop height and width can be quantified. Loop areas (total and subtotal) areas can be quantified. Loop areas per time can be quantified. The location of max and min dPa/dt relative to the loop center can be quantified. The symmetry of max dPa/dt versus dPa/dt loop locations can be characterized. Tangents along the loop can be characterized. The presence, location, frequency, and magnitude of loop artifacts can be characterized. These loop-derived measurements, downstream secondary measurements, and others combine to reveal characteristics of arterial pressurization and depressurization forces with each heartbeat. Additional measurements of ventricular and arterial power, as well as downstream blood flow and resistance, can also be quantified.

There are numerous commercially available devices that have the ability to measure or estimate arterial pressure waveforms and report such data as traditional time-frequency plots. These systems lack pressure loop analysis functionality.

FIG. 8 illustrates an example of standard aortic blood pressure tracing displayed by commercially available hemodynamic systems. Overall measurements include with systolic blood pressure, diastolic blood pressure, and mean arterial pressure.

FIG. 9 illustrates an example of an arterial “pressure loop” produced by the disclosed subject matter that is performed for numerous consecutive heart beats. The loops generated from these heartbeats are shown overlaid upon each other (using the same raw data source as FIG. 8). The black line is an average calculated from all of the loops calculated for each heartbeat. The left and right borders indicate points of minimum diastolic pressure and maximum systolic pressure, respectively. The top border and bottom border amplitudes represent maximum systolic dP/dt and minimum diastolic dP/dt, respectively.

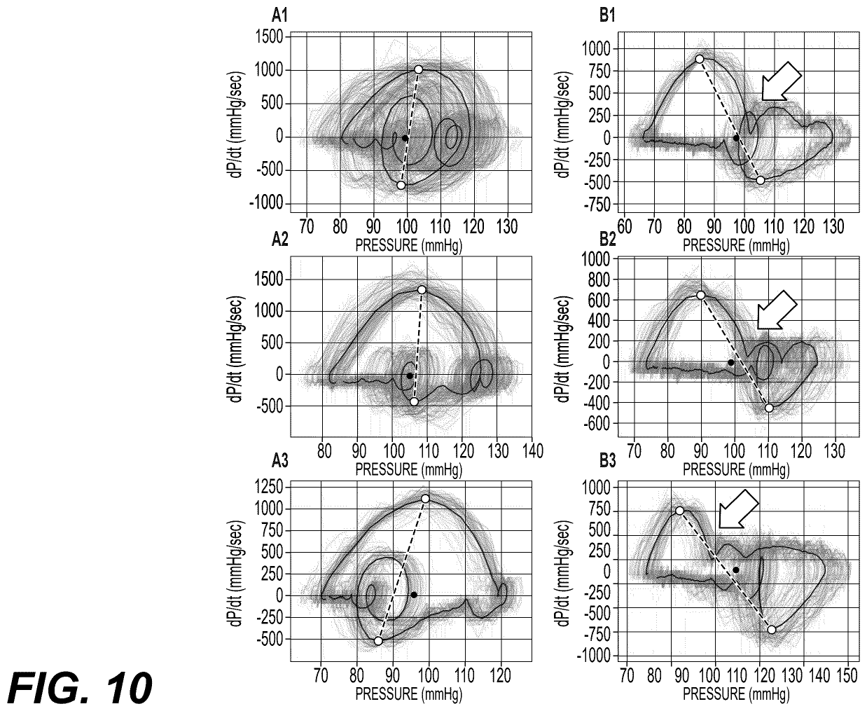

FIG. 10 illustrates arterial pressure loop variants produced by the disclosed subject matter. These variants demonstrate characteristics of arterial blood pressurization. Raw loops are shown as well as an overall average (black line). Loops in column A appear to have upper half curves that are “smooth” and “symmetric” compared to those in column B that appear “slanted” and “notched.” The point locations of max/min dPa/dt (black dots) appear to “rotate” around the pressure median (red dot) in some panels (row 1), but are “shifted” off the pressure mean in others (row 2 rightward shift, row 3 leftward shift). Rotation can occur either clockwise (column A) or counterclockwise (column B). In both columns, the 1) distance of max/min dPa/dt points from the X-axis and 2) area of the loop halves above/below the X-axis are variable. The relatively linear portion of each loop (lower left quadrant) demonstrates a “wave-free segment” with relatively diminished/absent cyclic pressure fluctuations.

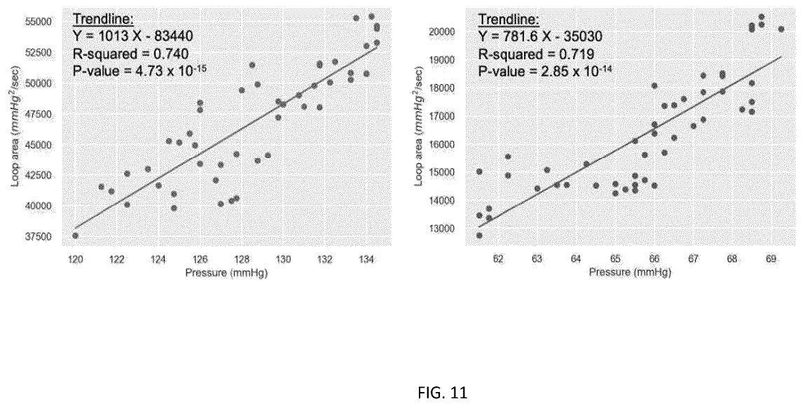

FIG. 11 illustrates simultaneous comparison of arterial pressure loop areas versus arterial pressures. Comparisons between total pressure loop area versus maximum Pa pressure (left panel) and lower pressure loop area versus minimum Pa pressure (right panel) are performed across multiple cardiac cycles. A significant linear relationship is demonstrated for each comparison and trendline shown.

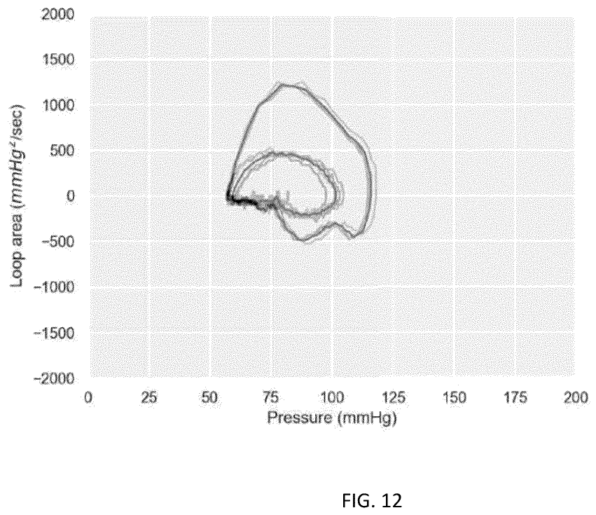

FIG. 12 illustrates a comparison of pressure loops produced from the same artery at different time points. The smaller inner loop is related to abnormal cardiac function, while the larger outer loop is related to normal cardiac function, both mirroring ventricular pressure loops in FIG. 5.

### Machine Learning

The pressure loop plots relating to both ventricular and arterial pressure may be analyzed by a machine learning model. The machine learning algorithm may be a neural network. The neural network may be, for example, a convolutional neural network.

The machine learning model may learn features of the images in order to screen for heart-related abnormalities. The machine learning model may be provided with training data comprising various sets of pressure loop plots from various patients, exhibiting healthy hearts and hearts with abnormalities. The model may use this data to associate particular combinations of features with healthy hearts and particular combinations of features with unhealthy hearts, for example.

The machine learning model may comprise a binary classifier. The binary classifier may be able to determine whether a heart in an image is healthy or whether there is an abnormality present. In some cases, the binary classifier may be used to predict the presence or absence of a particular abnormality. In some cases, the machine learning model may comprise a multiclass classifier, which may determine whether the heart includes an abnormality or whether the heart has one of several abnormalities. In some cases, the machine learning model may comprise a multilabel classifier, which may assign at least one abnormality label to a pressure loop plot.

### Arterial Pressure Decay Constant Tau

### Background:

Disclosed is a method for determining a time constant. The method comprises (a) taking one or more continuous waveform recordings of arterial pressure and dividing the one or more recordings into individual heartbeat cycles; (b) for each individual heartbeat cycle, converting the arterial pressure data into a time rate of change of arterial pressure; (c) determining a rolling standard deviation for each set of dP/dt data, revealing a period of diastole with minimal variation of dP/dt values; and (d) determining a set of values below a threshold to define a timing range for possible arterial pressure values to use to calculate the time constant.

Natural or exponential decay, wherein a quantity of something decreases at a rate proportional to its current amount, is commonly observed and calculated in various scientific fields. Its observation in cardiovascular medicine includes the manner in which blood pressure decreases within the cardiac ventricles and downstream arteries. The rate of pressure decay is quantified by the natural decay time constant Tau (Equations C1-2). In this field of study, Tau has been previously calculated for the purposes of 1) quantifying ventricular chamber relaxation using continuous ventricular pressure (Pv) measurements, 2) quantifying arterial vascular compliance (Equation C3), and 3) quantifying distal venous pressure (Equation C4) using continuous arterial pressure (Pa) measurements. These calculations have also been performed using non-invasive estimations of cardiovascular pressure using the ultrasound Doppler technique.

Tau has not been previously used for the purposes of evaluating other features of cardiovascular function, including vascular resistance, cardiac output, observed-to-expected arterial pressurization performance, or severe obstruction between the ventricular chamber and artery (i.e., aortic valve stenosis). The disclosed subject matter aims to perform these secondary evaluations based on Pa-derived Tau. While other techniques have attempted to use continuous Pa measurements to perform similar evaluations of cardiovascular function (Equations B6-10), none use Tau.

The disclosed subject matter also calculates Tau in a fashion by identifying the diastolic time period with minimal cyclic pressure fluctuations, which is not how Tau has been previously calculated. For this proprietary measurement, specifically, the ratio of distal to proximal pressures is quantified during the diastolic “wave-free period.” The calculation of Tau using this technique is not performed by this commercially-available product.

### Relevant Equations:

$$T = \frac{t}{ln ( \frac{P ( t )}{{P}_{0}} )} \qquad Equation C1$$

$$P ( t ) = {P}_{o} {e}^{- t / T} \qquad Equation C2$$

$$Resistance \times Compliance \qquad Equation C3$$

$$P ( t ) = ( {P}_{o} - C ) {e}^{- t / T} + C \qquad Equation C4$$

$${lim}_{t \to ∞} P ( t ) = C \qquad Equation C5$$

Calculation of unknown Tau and decay curve asymptote “C” values from Equation C4 can be performed by the disclosed subject matter by fitting an exponential curve to a set of data points. The disclosed subject matter can also perform this calculation using the following unique method and equations.

An equation incorporating the differences between Equation C2 using “raw” Tau and Equation C4 (values from “corrected” Tau) is established (Equation C6). For simplicity, “pressure curve 1” uses raw Tau and “pressure curve 2” uses corrected Tau. P(t) value at diastolic time 0 is represented by “P” for both curves. The pressure value at time “t” for curve 1 is represented by P₁(t). The pressure value at time “t” for curve 2 is represented by P₂(t). TR is “raw” Tau (known value), T_{C} is “corrected” Tau (to be calculated), and C is the decay curve asymptote (to be calculated).

$${P}_{1} ( t ) = {P}_{o} {e}^{- t / {T}_{R}} and {P}_{2} ( t ) = ( {P}_{0} - C ) {e}^{- t / {T}_{C}} + C$$

$${P}_{1} ( t ) - {P}_{2} ( t ) = {P}_{o} {e}^{- \frac{t}{{T}_{R}}} - [ ( {P}_{o} - C ) {e}^{- \frac{t}{{T}_{C}}} + C ]$$

$${P}_{1} ( t ) - {P}_{2} ( t ) = {P}_{o} {e}^{- \frac{t}{{T}_{R}}} - ( {P}_{o} {e}^{- \frac{t}{{T}_{C}}} - C {e}^{- \frac{t}{{T}_{C}}} + C )$$

$${P}_{1} ( t ) - {P}_{2} ( t ) = {P}_{o} {e}^{- \frac{t}{{T}_{R}}} - {P}_{0} {e}^{- \frac{t}{{T}_{C}}} + C {e}^{- \frac{t}{{T}_{C}}} - C$$

$${P}_{1} ( t ) - {P}_{2} ( t ) = {P}_{o} {e}^{- \frac{t}{{T}_{R}}} - {P}_{0} {e}^{- \frac{t}{{T}_{C}}} + C ( {e}^{- \frac{t}{{T}_{C}}} - 1 )$$

$$C ( {e}^{- \frac{t}{{T}_{C}}} - 1 ) = {P}_{1} ( t ) - {P}_{2} ( t ) - {P}_{o} {e}^{- \frac{t}{{T}_{R}}} + {P}_{0} {e}^{- \frac{t}{{T}_{C}}}$$

$$C = \frac{( {P}_{1} ( t ) - {P}_{2} ( t ) - {P}_{o} {e}^{- \frac{t}{{T}_{R}}} + {P}_{0} {e}^{\frac{t}{{T}_{C}}} )}{( {e}^{- \frac{t}{{T}_{C}}} - 1 )} \qquad Equation C6$$

For time point t_{A} and corresponding curve points (t_{A}, P₁(t_{A})) and (t_{A}, P₂(t_{A})),

$$C = \frac{( {P}_{1} ( {t}_{A} ) - {P}_{2} ( {t}_{A} ) - {P}_{o} {e}^{- \frac{{t}_{A}}{{T}_{R}}} + {P}_{0} {e}^{- \frac{{t}_{A}}{{T}_{c}}} )}{( {e}^{- \frac{{t}_{A}}{{T}_{c}} - 1 )}} \qquad Equation C7$$

For time point t_{B} and corresponding curve points (t_{B}, P₁(t_{B})) and (t_{B}, P₂(t_{B})),

$$C = \frac{( {P}_{1} ( {t}_{B} ) - {P}_{2} ( {t}_{B} ) - {P}_{o} {e}^{- \frac{{t}_{B}}{{T}_{R}}} + {P}_{0} {e}^{\frac{{t}_{B}}{{T}_{C}}} )}{( {e}^{- \frac{{t}_{B}}{{T}_{C}}} - 1 )} \qquad Equation C8$$

The values of C and T_{C} producing a curve (Equation C₄) that passes through points (t₀, P₀), (t_{A}, P₂(t_{A})) and (t_{B}, P₂(t_{B})) is then determined by finding the intersection of Equations C7 and C8.

$$Cardiac Output = \frac{Pressure gradient}{Tau} \times K \qquad Equation C9$$

$$Cardiac Output = \frac{Mean pressure - Asymptote pressure}{Corrected Tau} \times K \qquad Equation C10$$

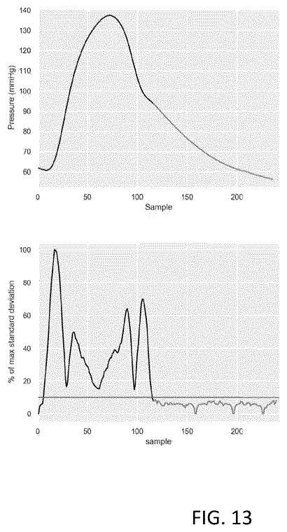

The disclosed subject matter automatically performs a primary calculation of arterial Tau based on the period of minimal cyclic pressure fluctuations, that is guided by user input settings and loop-derived limits of diastole. The automatic calculation of Tau involves multiple steps (FIG. 13) and begins by taking one or more continuous Pa waveform recordings and dividing them into individual heartbeat cycles. This can be performed by using a common start trigger, such as the R-wave of an electrocardiogram or the point of maximum dPa/dt. Pa data for each beat is converted to its first derivative dP/dt. A rolling standard deviation is calculated for each set of dP/dt data revealing a period of diastole with minimal variation of dP/dt values. The timing of standard deviation values below a prespecified threshold (e.g., lowest 10% of standard deviation values) then define the timing range for possible Pa values used for raw Tau calculation. Limits of the timing range and the corresponding Pa values are identified to calculate Tau (Equation C1). Because of variation in data sampling rates used to create Pa waveforms, actual time intervals rather than sample numbers should be used to calculate Tau.

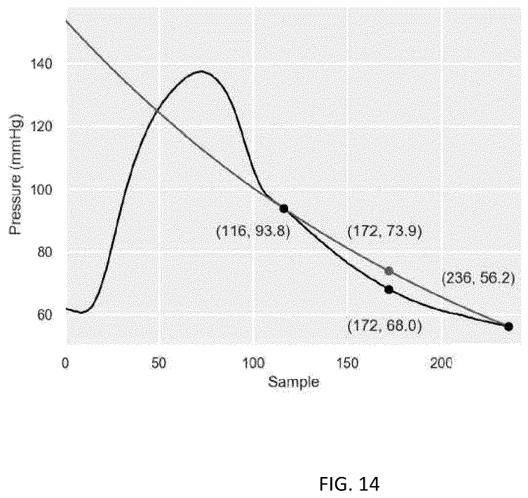

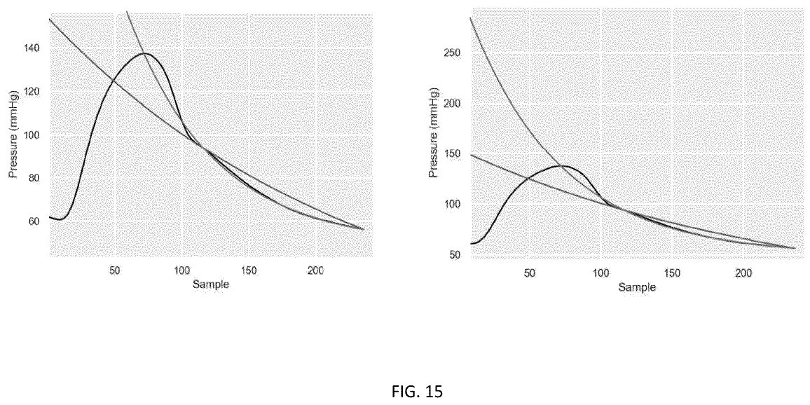

Once “raw” Tau has been calculated, the disclosed subject matter also calculates a “corrected” Tau to compensate for a non-zero decay curve asymptote (Equations C4 and C5). The process for calculating “corrected” Tau involves either 1) calculating the formula of a best-fit exponential curve for the data using widely-available methods, or 2) comparing observed Pa values versus calculated Pa values based on “raw” Tau (Equation C2) (FIG. 14). In the latter method, the timing and pressures at the point of maximum difference between the raw Tau pressure curve and actual Pa waveform are then used to calculate “corrected” Tau and the decay curve asymptote (FIG. 15). Using corrected Tau and the decay curve asymptote values in Equation C4 produces a pressure curve that closely approximates the actual diastolic Pa waveform.

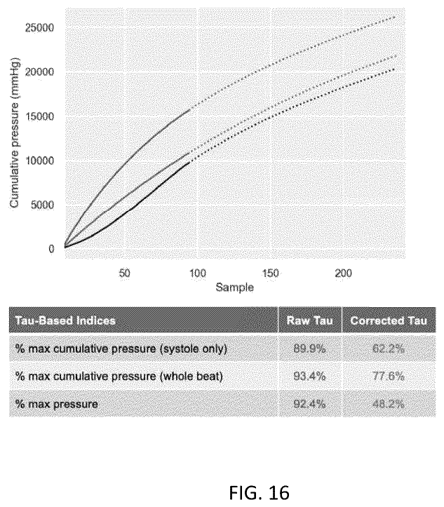

Raw and corrected Tau values can be used for additional secondary calculations including 1) backward calculation of expected mono-exponential curve during systole, and 2) calculation of cardiac output. First, the disclosed subject matter can calculate the monoexponential pressure curve that would occur during cardiac diastole and systole and compare actual versus calculated pressures. This analysis is performed from the beginning of systole to the end of diastole for each cardiac cycle. From this setup various comparisons for different waveform periods can be arranged, including percent maximum pressure, percent mean pressure, and/or percent cumulative pressure vs systole, diastole, and/or entire heartbeat (FIG. 16). Second, since Tau and arterial resistance are directly related (according to Equation C3), Tau may be used in place of resistance in Equation B1 (and similarly in Equations B2, B3 and B4) to create Equation C9. A variant of this equation includes calculating the gradient between mean arterial pressure and the decay curve asymptote from Equations C₃-C₈, divided by the corresponding corrected Tau (Equation C10). Since Tau and resistance values are related, but not identical, a correction factor K is applied to Equations C9 and C10 to calculate cardiac output.

FIG. 13 illustrates a determination of diastolic period with minimal cyclic pressure fluctuations. A single arterial pressure waveform is shown (top, entire line) and the diastolic period with minimal cyclic pressure fluctuations identified (top figure, green line). This period is identified by performing a rolling calculating of the first derivative dP/dt for the Pa waveform (bottom figure, black line). Plotting this first derivative data reveals a period of minimal cyclic pressure fluctuations within diastole. A prespecified threshold of lowest 10% of standard deviation values (red line) defines this period, which occurs from data sample 116 to 236.

FIG. 14 illustrates a determination of mono-exponential curve asymptote by the disclosed subject matter. A curve using “raw” Tau (red line) is created and passes through points (116,93.8) and (236,56.2) within the diastolic period with minimum pressure fluctuations from FIG. 1. Calculated raw Tau is approximately 234. The maximum difference between the raw Tau curve and observed arterial pressure (black line) occurs at sample number 172, correlating with points (172,73.9) and (172,68.0) on each line, respectively. These point values and raw Tau are used to populate Equations C7 and C8.

FIG. 15 illustrates a determination of “corrected” Tau by the disclosed subject matter. Equations C7 and C8 are populated using point values and raw Tau from FIG. 14. Solving these equations produces a corrected Tau value and mono-exponential curve asymptote (Tau of 65 samples, 0.270 second, or 0.0045 minute, and asymptote of 49.2 mmHg, for the example beat). Inserting these values into Equation C4 produces a curve using corrected Tau that passes through points (116,93.8), (172,68.0), (236,56.2), and more closely follows the observed diastolic pressure waveform (green line) than the curve using raw Tau (red line).

FIG. 16 illustrates a comparison of calculated versus actual arterial pressures by the disclosed subject matter. Examples of measurements derived from diastolic Tau and back calculated systolic pressures include actual versus calculated percent maximum cumulative pressure and percent maximum pressure. Different measurement permutations include those calculated for systole alone (based on solid lines), the whole beat (based on dotted lines), raw Tau (red line and values), and corrected Tau (green line and values).

### Analysis for Obstruction of Blood Flow

Disclosed is a method. The method comprises (a) over multiple cardiac cycles, determining a set of arterial-to-ventricular systolic pressure ratios (AVPRs) by dividing post-obstruction pressure by pre-obstruction pressure for values occurring from systole; and (b) generating a plot comprising each of the set of arterial-to-ventricular pressure ratios.

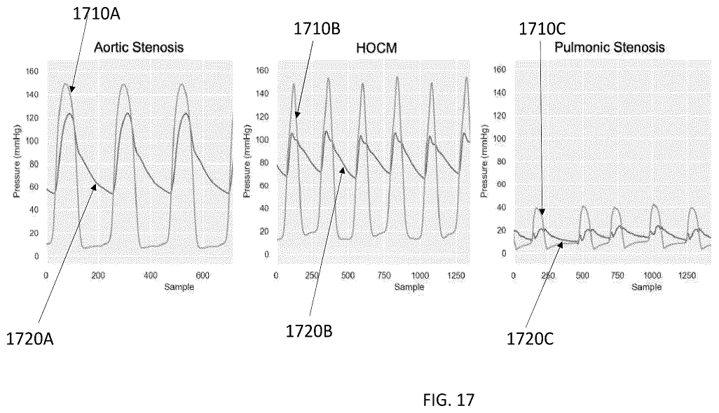

Evaluation of cardiac disease that causes obstruction of blood flow from a ventricle to its downstream artery is commonly performed. Pulmonic valve stenosis is a common reason for obstruction of right ventricular outflow to the pulmonary artery. Aortic valve stenosis (as well as supra-valvular and sub-valvular stenoses) and hypertrophic obstructive cardiomyopathy (HOCM) are common reasons for obstruction of left ventricular outflow to the aorta (FIG. 17). HOCM is caused by dynamic obstruction of left ventricular outflow due to abnormal interference by ventricular muscle and the mitral valve apparatus during ventricular contraction. For pulmonic valve stenosis, abnormally elevated transvalvular pressure gradients (by invasive and noninvasive techniques) and indices of detrimental right ventricular effects are used to determine the need for valve intervention and stenosis relief. For aortic valve stenosis, abnormally elevated transvalvular flow velocity and pressure gradient (by non-invasive echocardiography), and abnormally reduced aortic valve area (by both echocardiography and invasive valve study), are used to determine the need for valve intervention and stenosis relief. For HOCM, abnormally elevated flow velocity and pressure gradient across the obstruction (by non-invasive echocardiography), and/or abnormally elevated pressure gradient (by invasive pressure measurements), are used to determine the need for obstruction intervention and relief.

In particular, an aortic valve study utilizes invasive measurements from 1) right heart catheterization, and 2) left heart catheterization with simultaneous pressure measurements across the stenotic valve, to calculate aortic valve area using the Gorlin equation (Equation D1) or Hakki equation (Equation D2). Also, any of the aforementioned measurements can be performed with the patient at different physiologic states, including baseline rest, exercise, or pharmacologic stress. Discrimination between relatively static flow resistance due to aortic valve stenosis versus dynamic resistance due to HOCM is typically evaluated using echocardiography. Invasive hemodynamic evaluation of HOCM versus aortic stenosis can only crudely differentiate between the two etiologies.

The disclosed subject matter aims to provide an analysis of obstruction from a cardiac ventricle to its downstream artery to quantify the degree of obstruction present, characterize the static/dynamic nature of the obstruction, and characterize its effects on ventricular and arterial blood pressurization. The disclosed subject matter's method for doing so are described below.

$$( Gorlin ) := \frac{Cardiac output}{SEP \times HR \times 44.3 \times \sqrt{mean pressure gradient}} \qquad Equation D1$$

$$\begin{matrix}Equation D2 ( Hakki ) & \\ AVA = \frac{Cardiac output}{\sqrt{mean aortic valve pressure gradient}} & \end{matrix}$$

$${AVPR}_{M e a n} = \frac{{\int }_{T systole start}^{T systole end} \frac{{P}_{p ost - obstruction}}{{P}_{pre - obstruction}} d T}{T systole start - T systole end} \qquad Equation D3$$

$$Estimated {AVPR}_{M e a n} = \frac{MAP}{MAP + mean gradient} \qquad Equation D4$$

$$Estimated Fractional {Resistance}_{M e a n} = \frac{MAmean gradient}{MAP + mean gradient} \qquad Equation D5$$

The disclosed subject matter performs analysis of ventricular-to-arterial obstruction by 1) calculating and displaying arterial-to-ventricular systolic pressure ratios (AVPR) across the obstruction, 2) characterizing beat-by-beat pressure ratio measurements, 3) characterizing ventricular blood pressurization, and 4) characterizing arterial blood pressurization. These functions can be performed for any type of flow obstruction located between a cardiac ventricle and its downstream artery. These obstructions include, but are not limited to, pulmonic valve stenosis, aortic valve stenosis, HOCM, and arterial narrowing. Furthermore, the disclosed subject matter performs these analyses by analyzing simultaneous invasive blood pressure measurements upstream and downstream to the stenosis. These analyses can also be performed with the patient at resting baseline and repeated during cardiac stress with exercise or drugs. Additional invasive right heart catheterization is not needed for the disclosed subject matter's analyses.

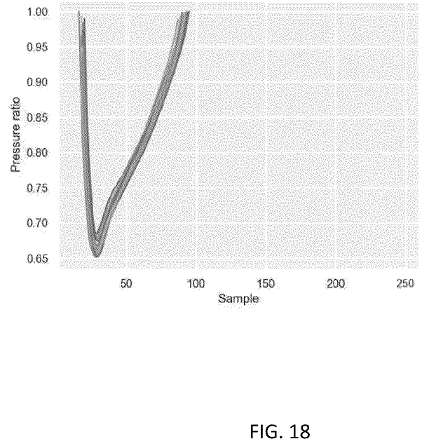

Systolic AVPR is produced from multiple calculations of post-obstruction pressure (lower value) divided by pre-obstruction pressure (higher value) for any values occurring during systole (identified when pre-obstruction pressure >post-obstruction pressure) (Equation D3). AVPR calculated within each systolic period can be graphically displayed by the disclosed subject matter and compared versus results from different cardiac cycles (FIGS. 18-21). These repeated measurements can also be displayed for a comparison across time (FIG. 4, row 2) and/or analyzed together (FIG. 4, table). AVPR measurements can also be compared with simultaneous ventricular and arterial pressure loop measurements (FIG. 5) in order to report the severity of ventricular-to-arterial obstruction as well as cardiac function, both of which contribute to the pressure gradients observed across blood flow obstructions.

The disclosed subject matter also may use a shorthand process for approximating the systolic AVPR (Equation D3). This shorthand method can also be used to calculate the “fractional resistance” of ventricular-to-arterial obstruction (Equation D4). Overall, this shorthand method simplifies the calculation and averaging of instantaneous AVPR by utilizing 1) the mean gradient across the obstruction and 2) the mean arterial pressure commonly measured by invasive and non-invasive methods. For pulmonic stenosis, estimated AVPR would be the quotient of mean pulmonary artery pressure (mPAP) divided by the sum of mPAP plus mean pulmonic valve gradient. For aortic stenosis, estimated AVPR would be the quotient of mean arterial pressure (MAP) divided by the sum of MAP and mean aortic valve gradient. For HOCM, estimated AVPR would be the quotient of MAP divided by the sum of MAP and mean left ventricular outflow tract gradient. In each of these situations, estimated fractional resistance due to the obstruction would be the mathematical compliment of AVPR.

This disclosure aims to protect the method that the disclosed subject matter calculates both exact AVPR (Equation D3) and estimated AVPR (Equation D4), as well as their mathematical compliments known as “fractional resistance.” Furthermore, AVPR is a unitless value that conceptually reflects the reduction in blood flow due to the obstruction, and its “fractional resistance” compliment is a unitless value that conceptually reflects the flow resistance due to the obstruction compared to other sources of resistance within the same blood flow circulation. Thus, if exact measures of blood flow or resistance were known, then AVPR and its complement could be used to calculate the absolute resistance due to the obstruction (FIG. 4 table). This disclosure also aims to protect such downstream calculations that would arise from first calculating AVPR by the disclosed subject matter.

For the disclosed subject matter, 1) cardiac stress testing with exercise or a pharmaceutical agent is permissible, but not necessary, for the disclosed subject matter's analysis, 2) multiple other functions in addition to arterial-to-ventricular pressure ratio calculation (none of which are described in the aforementioned manuscript) are performed by the disclosed subject matter, and 3) analysis by the disclosed subject matter is not limited to study of a stenotic aortic valve, but extends to any other conditions causing a ventricular-to-arterial obstruction (e.g., HOCM obstructing blood flow through the left ventricular outflow tract, and pulmonic valve stenosis obstructing blood flow from the right ventricle).

The disclosed subject matter's method of calculating AVPR is remotely similar, but significantly different, to “fractional flow reserve” (FFR) measurement, which is performed to quantify the severity of obstruction due to a coronary artery stenosis. FFR is calculated as the mean ratio of distal post-obstruction coronary artery pressure divided by proximal pre-obstruction aortic pressure throughout all parts of the cardiac cycle. FFR must also be performed during peak blood flow state induced by a coronary vasodilator drug such as adenosine. AVPR differs in 1) location of pressure measurements outside of the coronary artery, 2) ability to be measured at any physiologic state (including baseline rest, drug-induce peak blood flow, and exercise), 3) specific measurement during the systolic period rather than the entire cardiac cycle, and 4) simultaneous ventricular and arterial pressure loop analyses.

FIG. 17 illustrates simultaneous pressure tracings from ventricular (1710A-C) and downstream arterial (1720A-C) pressure sources reveal systolic pressure gradients due aortic valve stenosis (left chart), hypertrophic obstructive cardiomyopathy (HOCM: middle chart), and pulmonic valve stenosis (right chart). Pressure measurements from the left heart (left ventricle and aorta) and right heart (right ventricle and pulmonary artery) differ drastically in amplitude, but can both be utilized by the disclosed subject matter to analyze a degree of blood flow obstruction due to valve disease.

FIG. 18 illustrates an arterial-to-ventricular pressure ratio produced by the disclosed subject matter during valve stenosis. The disclosed subject matter identifies multiple cardiac cycles during within period of simultaneous pressure measurement (FIG. 18) in order to calculate and display systolic arterial-to-ventricular pressure ratios in this example of aortic valve stenosis. The minimal pressure ratio occurs regularly around the 30^{th} sample with consistent degree of obstruction observed between different cardiac cycles. Evaluation of pulmonic valve stenosis can be similarly performed.

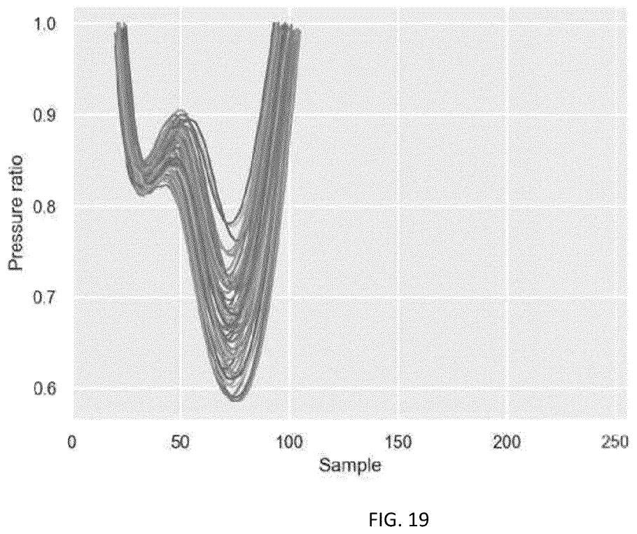

FIG. 19 illustrates an arterial-to-ventricular pressure ratio produced by the disclosed subject matter during hypertrophic obstructive cardiomyopathy. Multiple cardiac cycles are analyzed using simultaneous pressure measurements in order to calculate and display systolic arterial-to-ventricular pressure ratios (AVPR). Compared to the smooth curve produced during aortic stenosis (FIG. 19), analysis of hypertrophic obstructive cardiomyopathy shows a dynamic obstruction with a curve indentation occurring around the 50^{th} sample and variable degree of obstruction observed between different cardiac cycles. This graphical analysis may also support the finding of two source of obstruction; the first obstruction can be due to asymmetric septal hypertrophy occurring at the 30^{th} sample associated with higher (and more consistent) average AVPR, while the second obstruction can be due to systolic anterior motion of the mitral valve leaflet occurring at the 75^{th} sample associated with lower (and less consistent) average AVPR.

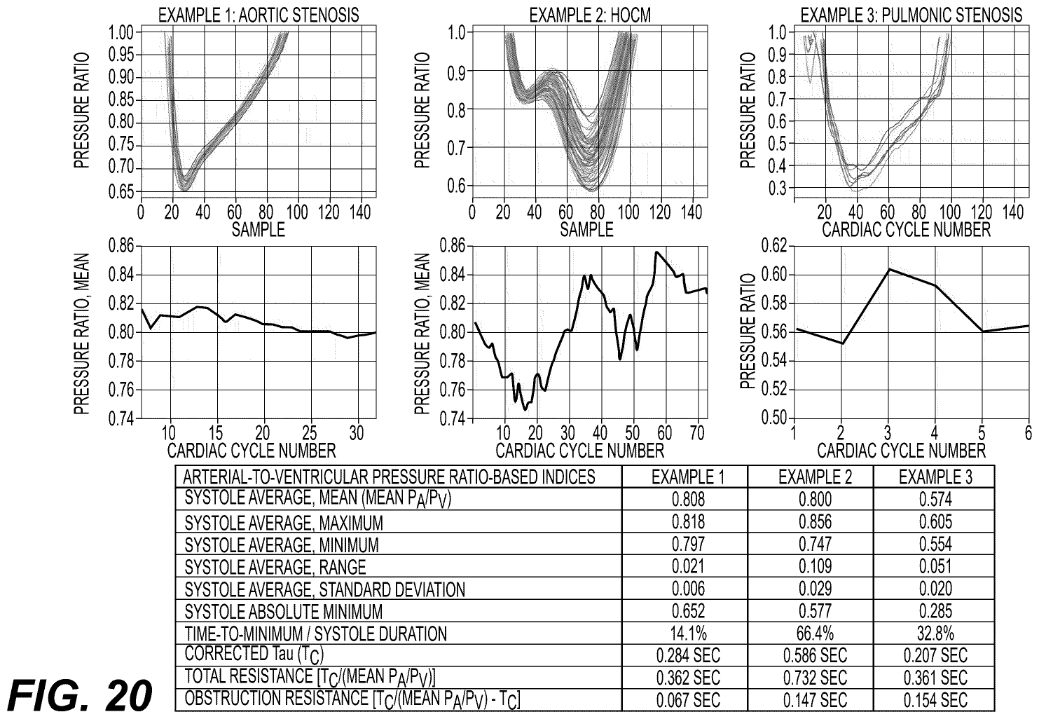

FIG. 20 illustrates arterial-to-ventricular pressure ratio indices produced by the disclosed subject matter. The disclosed subject matter quantifies and facilitates characterization of the arterial-to-ventricular pressure ratio (AVPR) produced from simultaneous arterial and ventricular pressure measurements. Graphical display allows visualization of overlapping cardiac cycles and AVPR calculations (row 1) to evaluate different disease conditions such as aortic valve stenosis (column 1), hypertrophic obstructive cardiomyopathy (HOCM; column 2), and pulmonic valve stenosis (column 3). Stability or instability of AVPR across multiple cardiac cycles can be graphically displayed (row 2) that can be modified by different provocative techniques/agents and tracked over time. Different possible indices include, but are not limited to, average AVPR (e.g., minimum, maximum, mean, range, standard deviation across multiple beats), minimum AVPR across multiple beats, and timing of minimum AVPR within the systolic period (table). Tau calculations (e.g., from Equations D1-4) can be performed using AVPR. Notably, these analyses are performed from either left heart (aorta and left ventricle, Examples 1 and 2) or right heart (pulmonary artery and right ventricle, Example 3) pressure measurements.

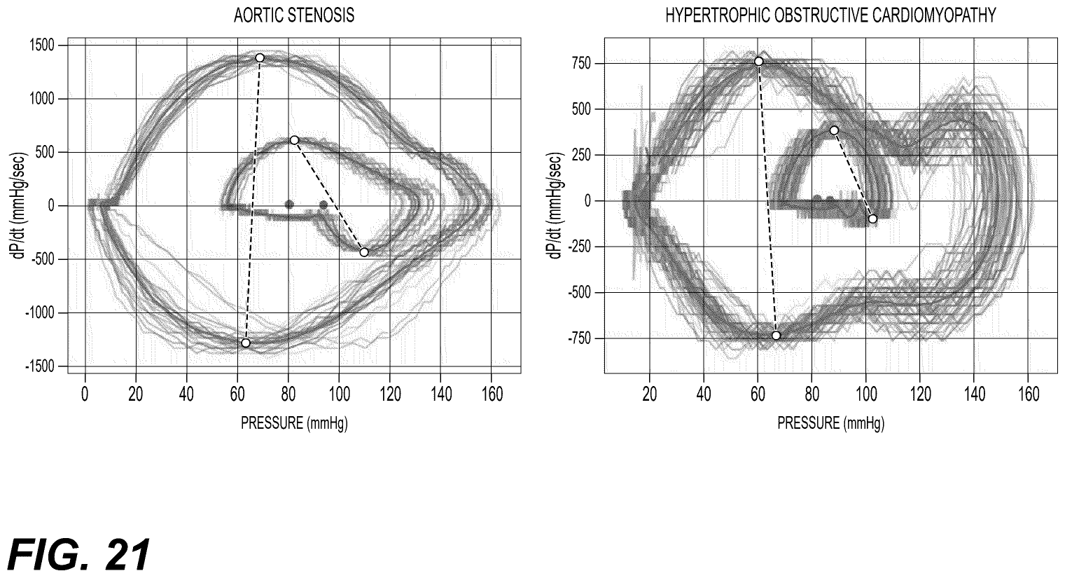

FIG. 21 illustrates a novel comparison of multiple pressure loops produced by the disclosed subject matter. Multiple cardiac cycles are analyzed to produce ventricular pressure loops (2110A-B), and arterial pressure loops (2120A-B). Comparison of ventricular and arterial pressure loops between the left chart (example of aortic stenosis) and right chart (example of hypertrophic obstructive cardiomyopathy) reveals drastically different loop size and shape among other characteristics. In some cases, AVPR data may be combined with pressure loop data. A combination of AVPR and pressure loop data may be displayed together in an electronic report (e.g., as visual objects in a graphical user interface displayable on a computer screen). The electronic report may comprise a three-dimensional (3D) plot including both the pressure loop and AVPR data.

### Analysis of Fractional Resistance

Disclosed is a method for determining a fractional resistance by (a) determining a segmental gradient associated with a portion of a heart; (b) determining a total gradient associated with the portion of the heart; and (c) dividing the segmental gradient by the total gradient.

Quantification of blood flow resistance between an upstream artery and a downstream vein is routinely performed as part of an invasive evaluation of cardiovascular hemodynamics. Lung conditions leading to pulmonary vascular disease are reflected in abnormally elevated pulmonary vascular resistance (PVR), while systemic conditions leading to systemic vascular disease within the body are reflected in abnormally reduced or elevated systemic vascular resistance (SVR).

The evaluation of lung disease and PVR is commonly performed by performing right heart catheterization (RHC). RHC allows for measurement of cardiac output, central venous pressure (CVP), right ventricular pressure, pulmonary artery pressure (PAP) and pulmonary capillary wedge pressure (PCWP). PCWP is determined by “wedging” a small balloon within a pulmonary artery branch, thereby occluding blood flow and allowing measurement of pressure distal to the occlusion. Secondary calculation of PVR is routinely performed from these RHC measurements (Equation E1). PVR is measured in Wood units, which can be multiplied by 80 and converted to dynes-seconds/cms, and is an absolute (rather than relative) measure of pulmonary blood flow resistance. An existing variation of PVR is the PVR index, which normalizes PVR value according to patient body surface area (Equation E2). Disease conditions that are associated with abnormally elevated PVR include emphysema, pulmonary fibrosis, and pulmonary thromboembolic disease.

The evaluation of systemic disease affecting arterial blood pressure and SVR is commonly performed by combining some RHC measurements (CVP and cardiac output) with an additional measurement of mean arterial pressure (Equation E3). SVR is also measured in Wood units, converted to dynes-seconds/cms, and normalized by patient body surface area (Equation E4). Reduced SVR is associated with disease conditions such as infection and vasoplegia, while elevated SVR is associated with disease conditions such as hypertension, hypovolemia, and congestive heart failure.

### Relevant Equations:

$$PVR = \frac{mPAP - PCWP}{cardiac output} \qquad Equation E1$$

$$PVR index = \frac{PVR}{body surface area} \qquad Equation E2$$

$$SVR = \frac{MAP - CVP}{cardiac output} \qquad Equation E3$$

$$SVR index = \frac{SVR}{body surface area} \qquad Equation E4$$

$$\frac{segmental resistance}{total resistance} = \frac{( \frac{segmental pressure gradient}{cardiac output} )}{( \frac{total pressure gradient}{cardiac output} )} \qquad Equation E5$$

$$\frac{segmental resistance}{totalresistance} = \frac{segmental pressure gradient}{total pressure gradient}$$

$$fractional resistance = \frac{segmental pressure gradient}{total pressure gradient} \qquad Equation E6$$

For PVR or SVR estimation, Pa measurements would be obtained from the proximal pulmonary artery or aorta, while Pd measurements would be obtained from the pulmonary vein (PCWP) or systemic vein (CVP), respectively. Equations E7 and E8 provide specific examples for calculation pulmonary and systemic fractional resistance.

$$pulmonary vascular fractional resistance = \frac{mPAP - PCWP}{mPAP} \qquad Equation E7$$

$$systemic vascular fractional resistance = \frac{MAP - CVP}{MAP} \qquad Equation E8$$

mPAP=mean pulmonary artery pressure; PCWP=pulmonary capillary wedge pressure; MAP=mean arterial pressure; CVP=central venous pressure

The disclosed subject matter performs analysis of blood flow obstruction between an upstream artery and a downstream vein that is uniquely different from that of PVR or SVR. According to Equations E1 through E4, a pressure gradient that exists within a vascular segment is directly proportional to the vascular resistance present, while gradients of different amplitudes are typically normalized against cardiac output and BSA to facilitate intercomparison. The disclosed subject matter uniquely calculates “fractional resistance,” which is 1) a measure of resistance through a specific portion/segment of a cardiovascular flow circuit and 2) normalized by total resistance through the entire circuit. This calculation is derived by relating a ratio of segmental and total resistances with that of pressure gradients (Equation E5), based on Equations E1 and E3. Thus, a “fractional resistance” calculation is the ratio of a segmental pressure gradient and its corresponding total pressure gradient (Equation E6). In doing so, the disclosed subject matter normalizes a pressure gradient against other high-fidelity pressure measurements rather than other potentially problematic measures (cardiac output and body surface area). The disclosed subject matter's fractional resistance calculation 1) provides surrogate measurements to traditional PVR and SVR (Equations E7 and E8), respectively, 2) quantify PVR and SVR as relative, unitless measurements, and 3) facilitates comparison of values across different cardiac output states, body sizes, and individuals. The calculation of such a fractional resistance measurement has never been previously described and is not commercially available. Beyond estimating PVR and SVR, the disclosed subject matter's analysis can also be used to calculate the fractional resistance of other hemodynamic obstructions as listed below:

### Fractional Resistance Between Right Ventricle and Left Ventricle:

Fractional resistance due to pulmonic valve stenosis:
   Segmental gradient=systolic right ventricular pressure minus systolic pulmonary artery pressure.
   Total gradient=systolic right ventricular pressure minus zero.
Fractional resistance due to pulmonary artery stenosis (e.g., chronic pulmonary thromboembolic disease)
   Segmental gradient=mean pulmonary artery pressure (mPAP) proximal to thromboembolic disease minus mPAP distal to thromboembolic disease.
   Total gradient=mPAP proximal to thromboembolic disease minus zero.
Fractional resistance due to mitral valve stenosis between the left atrium and left ventricle
   Segmental gradient=diastolic PCWP minus diastolic left ventricular pressure.
   Total gradient=diastolic PAP minus diastolic left ventricular pressure.
Fractional resistance due to blood collection in the left atrium and left ventricle (e.g., congestive heart failure due to left ventricular dysfunction)
   Segmental gradient=PCWP minus zero.
   Total gradient=mPAP minus zero.

### Fractional Resistance Between Left Ventricle and Right Ventricle:

Fractional resistance due to aortic valve stenosis:
   Segmental gradient=systolic left ventricular pressure minus systolic aortic pressure.
   Total gradient=systolic left ventricular pressure minus zero.
Fractional resistance due to hypertrophic obstructive cardiomyopathy:
   Segmental gradient=systolic left ventricular pressure proximal to obstruction minus systolic left ventricular outflow tract distal to obstruction.
   Total gradient=systolic left ventricular pressure proximal to obstruction minus zero.
Fractional resistance due to arterial stenosis (e.g., coronary artery disease, peripheral artery disease, aortic coarctation)
   Segmental gradient=mean proximal arterial pressure minus mean distal arterial pressure.
   Total gradient=mean proximal arterial pressure minus zero.
Fractional resistance due to a systemic vein stenosis (e.g., thrombosed filter device in the inferior vena cava).
   Segmental gradient=mean distal venous pressure minus mean proximal venous pressure.
   Total gradient=mean arterial pressure minus zero.
Fractional resistance due to tricuspid valve stenosis
   Segmental gradient=diastolic CVP minus diastolic right ventricular pressure.
   Total gradient=diastolic arterial pressure minus diastolic right ventricular pressure.
Fractional resistance due to blood collection in the right atrium and right ventricle (e.g., congestive heart failure due to right ventricular dysfunction)
   Segmental gradient=CVP minus zero.
   Total gradient=MAP minus zero.

Fractional resistance in special circumstances: The disclosed subject matter can calculate fractional resistance values in special circumstances. One example is quantifying fractional resistance of the mitral valve during left ventricular contraction, which should be closer to a value of one during normal valve function, and closer to zero with more severe mitral regurgitation. A similar example is quantifying fractional resistance of the tricuspid valve during right ventricular contraction, which also should be closer to a value of one during normal valve function, and closer to zero with more severe tricuspid regurgitation. Fractional resistance in these circumstances can be calculated from mean or peak input values, and reported as peak or mean output values. Baseline left atrial pressure can be set as the level where ventricular and atrial pressures are equivalent at early systole, or any other baseline level.
Fractional resistance due to mitral valve regurgitation
   Segmental gradient=systolic left ventricular pressure minus non-baseline systolic left atrial pressure.
   Total gradient=systolic left ventricular pressure minus baseline left atrial pressure.
Fractional resistance due to tricuspid valve regurgitation
   Segmental gradient=systolic right ventricular pressure minus nonbaseline right atrial pressure.
   Total gradient=systolic right ventricular pressure minus baseline right atrial pressure.

It is anticipated that fractional resistance calculations are more reliable when 1) fewer sources of fractional resistance are present simultaneously, and 2) intracardiac shunting is absent or minimal. In the presence of multiple overlapping source of fractional resistance and significant intracardiac shunting, special calculations beyond the scope of this disclosure may be needed.

Existing products that perform “fractional flow reserve” (FFR) measurements in the coronary arteries utilize common underlying principles as the disclosed subject matter, but for multiple important reasons does not perform the same analysis. FFR is ratio of maximal achievable blood flow to maximal theoretical blood flow through an epicardial coronary artery. Using pressure measurements as a surrogate for blood flow. FFR is calculated as the ratio of distal pressure divided by aortic pressure (Pd/Pa) achieved during hyperemia and values <0.75-0.80 identify significantly reduced blood flow through the interrogated vessel segment. Resting Pd/Pa values without inducing hyperemic blood flow have also be used for the same purpose. Pd/Pa values are unitless and conceptually represent the fraction of maximal theoretical blood flow that occurs in the presence of a coronary artery stenosis. Relief of the coronary artery stenosis would theoretically improve FFR to its theoretical maximal value of 1.0. The values used to calculate FFR or resting Pd/Pa are not the same as those the disclosed subject matter uses to calculate fractional resistance.

Conceptually, CFR represents the scaling factor by which coronary blood flow increases during hyperemia versus during rest. While 1-Pd/Pa can be rearranged as (Pa−Pd)/Pa, mirroring the right-hand expression of Equation E6, existing methods must also create a ratio of (Pa—Pd)/Pa values occurring at hyperemia and at rest in order to produce its CFR measurement. The individual numerator and denominator elements of this previously-described ratio are 1) unique measurements of resistance rather than flow, 2) additive to one another when acting as resistance values in series, 3) applicable within all cardiovascular territories (not solely the coronary arteries), and 4) may undergo further adjustment when measured in the presence of a ventricle-to-arterial obstruction (e.g., pulmonic valve stenosis, aortic valve stenosis, HOCM), making them uniquely different from existing art and forming the basis of the disclosed subject matter.

### Analysis of Coronary Artery Resistance

Disclosed is a method comprising (a) determining a fixed epicardial resistance; (b) determining a fixed microvascular resistance; (c) determining an adenosine-responsive microvascular resistance; (d) determining a medication-responsive epicardial resistance; (e) determining a medication-responsive microvascular resistance; and (f) determining a total resistance of blood flow at least in part by determining a sum of the fixed epicardial resistance, the fixed microvascular resistance, the adenosine-responsive microvascular resistance, the medication-responsive epicardial resistance, and the medication-responsive microvascular resistance.

Resistance of blood flow through an entire coronary artery occurs within both large-caliber proximal epicardial segments and distal microscopic vasculature. While high-quality measurements of flow resistance due to epicardial coronary artery disease (CAD) exist, there is no high-quality measurement of flow resistance due to microvascular CAD. This is a description for a measurement of blood flow resistance due to coronary artery disease of both epicardial and microvascular coronary artery segments.

Quantification of reduced coronary blood flow due to epicardial CAD is most accurately and precisely performed by measuring proximal arterial pressure (Pa) and distal pressure (Pd) across the diseased segment at maximal hyperemia and calculating fractional flow reserve (FFR; Equation F1). FFR is also approximated using the ratio of resting, non-hyperemic, Pd/Pa using data from the entire heart beat cycle (Equation F2) as well as the diastolic “wave-free” period (Equation F3). Non-invasive techniques to estimate FFR, such as those using cineangiography or coherence tomography coronary angiography, have also been developed.

Quantification of reduced coronary blood flow due to microvascular CAD is approximated by calculating the ratio of hyperemic and resting coronary blood flow known as coronary flow reserve (CFR; Equation F4). While CFR is generally used to quantify the degree of microvascular CAD, its values are also influenced by epicardial CAD thus making CFR a non-specific metric of microvascular CAD. In the presence of severe epicardial CAD, abnormally reduced CFR due to concurrent microvascular CAD is difficult to accurately identify. All techniques of measuring CFR suffer from this limitation. These CFR techniques include coronary blood velocimetry, indicator thermodilution, non-invasive coronary blood flow imaging, and pressure-derived CFR (CFRp).

Specific measurements of microvascular CAD have been previously created based on simultaneous FFR and CFR values. The first measurement is Hyperemic Microvascular Resistance calculated as distal epicardial pressure (Pd) divided by average peak blood velocity measured during hyperemia (Equation F6). The second measurement is Index of Microvascular Resistance calculated as Pd multiplied by temperature curve mean transit time measured during hyperemia (Equation F7). The third measurement is myocardial flow reserve (MFR), calculated as the ratio of hyperemic myocardial blood flow divided by resting myocardial blood flow, and quantified by non-invasive positron emission tomography or cardiac magnetic resonance techniques.

The analysis of coronary artery resistance described in this section quantifies both epicardial and microvascular coronary artery resistance in manner that is distinct from all previously described techniques by 1) establishing a conceptual framework for identifying multiple sources of coronary artery resistance, 2) measuring the relative quantities of all coronary artery resistance sources, 3) determining the relative coronary artery resistance due to blood vessels that can dilate in response to the pharmacologic agent, adenosine, 4) determining the relative coronary artery resistance due to blood vessels that can contract or dilate in response to a non-adenosine pharmacologic agent (i.e., nitroglycerin, calcium channel blocker, other type of medication with effect on coronary artery resistance), 5) using both/only FFR and CFR measurements for this type of analysis, and 6) utilizing pressure-derived CFR in a unique manner. While other commercially-available devices and techniques are able measure both FFR and CFR, each of these aspects of the disclosed subject matter's analysis distinguish it from preexisting methods of quantifying microvascular coronary artery resistance.

### Relevant Equations:

$$FFR = \frac{{Pd}_{Hyperemia}}{P {a}_{Hyperemia}} \qquad Equation F1$$

$$Resting Pd / P a = \frac{{Pd}_{Rest}}{P {a}_{Rest}} \qquad Equation F2$$

$$iFR = \frac{{Pd}_{Rest , diastole only}}{P {a}_{Rest , diastole only}} \qquad Equation F3$$

$$CFR = \frac{{CBF}_{Hyperemia}}{{CBF}_{Rest}} \qquad Equation F4$$

$$CFRp = \frac{{( 1 - \frac{Pd}{P a} )}_{Hyperemia}}{{( 1 - \frac{Pd}{P a} )}_{Rest}} \qquad Equation F5$$

$$HMR = \frac{PD}{{APV}_{Hyperemia}} \qquad Equation F6$$

$$IMR = {( Pd \times {T}_{mn} )}_{Hyperemia} \qquad Equation F7$$

$$Flow = \frac{Pressure gradient}{Resistance} \qquad Equation F8$$

$${R}_{Fixed , Hyperemia} = {R}_{Fixed , Epi} + {R}_{Fixed , Micro} \qquad Equation F9$$

$${R}_{Total , Rest} = {R}_{Fixed , Epi} + {R}_{Fixed , Micro} + {R}_{Adenosine , Micro} \qquad Equation F10$$

$$CFR = \frac{{CBF}_{Hyperemia}}{{CBF}_{Rest}} = \frac{{R}_{Total , Rest}}{{R}_{Fixed , Hyperemia}} \qquad Equation F11$$

$$CFR = \frac{( {R}_{Fixed , Epi} + {R}_{Fixed , Micro} ) + {R}_{Adenosine , Micro}}{( {R}_{Fixed , Epi} + {R}_{Fixed , Micro} )}$$

$$CFR = \frac{( 1 ) + {R}_{Adenosine , Mirco}}{( 1 )}$$

$$CFR = 1 + {R}_{Adenosine , Micro}$$

$${R}_{Adenosine , Micro} = CFR - 1 \qquad Equation F12$$

$${R}_{Fixed , Micro} = FFR \qquad Equation F13$$

$${R}_{Fixed , Epi} = 1 - FFR \qquad Equation F14$$

$${R}_{Total , Rest} = CFR \qquad Equation F15$$

When solving for MER and MMR,

$${R}_{Total , PreMed , Rest} \times κ = {R}_{Total , PostMed , Rest}$$

$$κ = \frac{{CFR}_{PostMed}}{{CFR}_{PreMed}}$$

$${R}_{Total , PostMed , Rest} = {CFR}_{PostMed}$$

$${R}_{Total , PreMed , Rest} = ( 1 - {FFR}_{PreMed} ) + ( {FFR}_{PreMed} ) + {ARMR}_{PreMed}$$

$$κ [ ( 1 - {FFR}_{PreMed} ) + ( {FFR}_{PreMed} ) + {ARMR}_{PreMed} ] = {CFR}_{PostMed}$$

$${R}_{Total , PreMed , Rest} = ( {R}_{Med , Epi} + {R}_{Fixed , Epi} ) + ( {R}_{Med , Micro} + {R}_{Fixed , Micro} ) + {ARMR}_{PreMed}$$

$$( {R}_{Med , Epi} + {R}_{Fixed , Epi} ) = κ ( 1 - {FFR}_{PreMed} )$$

$$( {R}_{Med , Micro} + {R}_{Fixed , Micro} ) = κ ( {FFR}_{PreMed} )$$

$${R}_{Fixed , Epi} = 1 - {FFR}_{PostMed}$$

$${R}_{Fixed , Micro} = {FFR}_{PostMed}$$

$$Solving for MER : ( {R}_{Med , Epi} + {R}_{Fixed , Epi} ) = κ ( 1 - {FFR}_{PreMed} )$$

$${R}_{Med , Epi} = κ ( 1 - {FFR}_{PreMed} ) - {R}_{Fixed , Epi}$$

$${R}_{Med , Epi} = κ ( 1 - {FFR}_{PreMed} ) - ( 1 - {FFR}_{PostMed} )$$

$${R}_{Total , PreMed , Rest} = ( {R}_{Med , Epi} + {R}_{Fixed , Epi} ) + ( {R}_{Med , Micro} + {R}_{Fixed , Micro} ) + ARM {R}_{PreMed} \qquad Equation F16$$

$${R}_{Med , Epi} = κ ( 1 - {FFR}_{PreMed} ) - ( 1 - {FFR}_{PostMed} ) \qquad Equation F17$$

$${R}_{Med , Micro} = κ ( {FFR}_{PreMed} ) - {FFR}_{PostMed} \qquad Equation F18$$

$${R}_{Total , Med} = {R}_{Med , Epi} + {R}_{Med , Micro} \qquad Equation F19$$

| | Denominator | | | |
|---|---|---|---|---|
| Numerator | R_{Total} | R_{Fixed Total} | R_{Total, Micro} | R_{Fixed, Epi} |
| R_{Adenosine Micro} | ARMR/CFR | CFR − 1 | ARMR/(FFR + ARMR) | ARMR/(1 − FFR) |
| R_{Fixed Micro} | FFR/CFR | FFR | FFR/(FFR + ARMR) | FFR/(1 − FFR) |
| R_{Fixed, Epi} | (1 − FFR)/CFR | 1 − FFR | 1 − FFR)/(FFR + ARMR) | — |
| R_{TotalMicro} | (FFR + ARMR)/CFR | FFR + ARMR | — | FFR + ARMR)/(1 − |
| | | | | FFR) |
| R_{Total Fixed} | 1/CFR | — | 1/(FFR + ARMR) | 1/(1 − FFR) |
| R_{Med, Micro} | R_{Med, Micro}CFR | R_{Med, Micro} | R_{Med, Micro}(FFR + ARMR) | R_{Med, Micro}/{1 − FF R) |
| R_{Med, Epi} | R_{Med, Epi}/CFR | R_{Med, Epi} | R_{Med, Epi}(FFR + ARMR) | R_{Med, Epi}/(1 − FFR) |
| R_{Total Med} | R_{tOtal, Med}/CFR | R_{total, Med} | R_{Total, Meci}/(FFR + ARMR) | R_{total, Micro}/(1 − FFR) |
| Other . . . | | | | |

The disclosed subject matter performs its analysis of coronary artery resistance by first establishing a conceptual framework for identifying multiple sources resistance, followed by measuring the relative quantities of all coronary artery resistance sources. Doing so enables the disclosed subject matter and users to then evaluate coronary artery response to vasodilatory pharmacologic agents such as adenosine, nitroglycerin, calcium channel blockers, and others.

The disclosed subject matter's conceptual framework for coronary artery resistance is that total resistance is the sum of multiple different resistance subtypes. This concept is based on the inverse relationship between cardiovascular flow and resistance (Equations B1 and F8). At baseline resting state five resistance subtypes are identified:
1) fixed epicardial resistance (FER)
2) fixed microvascular resistance (FMR)
3) “adenosine-responsive” microvascular resistance (ARMR)
4) “medication-responsive” epicardial resistance (MER)
5) “medication-responsive” microvascular resistance (MMR)

Epicardial resistance is defined as that occurring proximal to the intracoronary pressure sensor used to measure FFR/CFRp. Microvascular resistance is defined as that occurring distal to the intracoronary pressure sensor. It is anticipated that pressure sensor positioning within a distal epicardial artery segment would be beneficial for the disclosed subject matter's analysis. Fixed resistance is that which remains after administration of vasodilatory pharmacologic agents. Adenosine-responsive resistance is that which is directly impacted after in-vivo administration of adenosine to induce maximal coronary hyperemia. Medication-responsive resistance is that which is directed impacted following administration of a vasodilatory medication other than adenosine; this is a general term to encompass the use of various coronary vasodilators, but admittedly the most commonly used agent during FFR/CFRp measurement is nitroglycerin.

Nitroglycerin is routinely administered before FFR/CFR measurements to dilate epicardial arteries and provide a baseline hemodynamic state to compare different measurements. Following the administration of a coronary vasodilator, such as nitroglycerin, MER and MMR are assumed to reduce to zero and the remaining sources of coronary resistance simplifies to include three resistance subtypes:
1. fixed epicardial resistance (FER)
2. fixed microvascular resistance (FMR)
3. “adenosine-responsive” microvascular resistance (ARMR) By measuring FFR and CFRp before and after administering a vasodilator medication, all give resistance subtypes can be calculated by the disclosed subject matter. By measuring FFR and CFRp only after administering a vasodilator medication only three resistance subtypes can be identified. By measuring FFR and CFRp without administering any vasodilator medication, three resistance subtypes can be approximated while the impact of medication-responsive resistance remains unknown.

In the simplest clinical scenario, a vasodilator medication is administered, then FFR and CFRp are measured. During adenosine-induced hyperemic blood flow, ARMR transiently reduces to zero and total fixed coronary resistance (TFR) equals the sum of FER and FMR (Equation F9). During resting blood flow, when ARMR returns to baseline level, total coronary resistance (TCR) equals the sum of FER, FRM, and ARMR (Equation F10). The ratio of TCR/TFR can be equated to CFR (Equation F11). By defining TFR as a relative value of 1, then Equation F11 can be simplified and ARMR defined (Equation F12). FMR and FER are then equated to FFR and the complement of FFR, respectively (Equations F13 and F14). From this, the first three subtypes of coronary artery resistance are quantified by the disclosed subject matter (Equations F12 to F14) and their sum is equated to the value of CFR (Equation F15).

In the next clinical scenario, FFR and CFRp are measured twice, once before and once after a vasodilator medication is administered. During hyperemic blood flow occurring before a vasodilator medication is administered, ARMR transiently reduces to zero and TFR equals the sum of FER, FMR, MER, and MMR. At baseline blood flow, TCR equals the sum of FER, FMR, MER, MMR, and ARMR (Equation F16). Since TCR is defined as an unchanging value, but also equated to the value of CFR that can change between measurements (Equations F10 and F15), a correction is used to keep TCR constant. Using the known values for FFR and CFR measured both before and after medication administration, the relative values for MER, MMR, and their total can be solved (Equations F17 to F19). The effects of various medications on coronary artery resistance, including nitroglycerin/nitrates, calcium channel blockers, beta blockers, and other classes of medications, can be evaluated using this method. Based on the disclosed subject matter's conceptual framework and method for determining total coronary artery resistance and each of its subparts, various specific and unique measurements of microvascular resistance can be produced (Table, not exhaustive). While many different combinations of resistance measures can be reported by the disclosed subject matter, it is unknown at this time which of these unique measurements of microvascular resistance produced by the disclosed subject matter will be most useful for clinical or research purposes. Three unique measurements reported by the disclosed subject matter worthy of consideration for patent protection include 1) ARMR/TFR, 2) TFR/CFR, and 3) total medication-responsive resistance.

First, ARMR/TFR indicates the quantity of adenosine-responsive resistance present relative to the total fixed resistance present. Higher ARMR/TFR may indicate good vascular health due to low epicardial and microvascular CAD burden and high “ARMR reserve”, while ARMR/TFR of zero may indicate poor vascular heath due to high CAD burden and/or zero ARMR reserve. While the numeric value of ARMR/TFR simplifies to CFR-1, the disclosed subject matter's conceptual framework imparts highly significant meaning to this basic, yet novel, calculation.

Second, TFR/CFR indicates the proportion of fixed coronary artery resistance relative to resting total coronary artery resistance. TFR/CFR close to zero may indicate a low relative burden of epicardial and microvascular CAD, while TFR/CFR of 1 indicates that all coronary artery resistance present at rest is due to the effects of CAD. Generally speaking, this metric can be converted/used to report “percent CAD burden” within an artery. While the numeric value of TFR/CFR simplifies to 1/CFR, the disclosed subject matter's conceptual framework imparts highly significant meaning to this basic, yet novel, calculation.

Third, total medication-responsive resistance and its epicardial/microvascular subparts are completely unique metrics reported by the disclosed subject matter. Positive medication-responsive resistance values indicate the quantity of resistance that is relieved by administration of a medication (due to a vasodilatory effect), while negative medication-responsive resistance values indicate resistance that is imparted by administration of a medication (due to a vasoconstrictive effect). The total medication-responsive resistance value indicates the overall effect of a medication on coronary artery resistance, and value of its subparts indicate the regionality and symmetry of this effect. This metric may potentially be used to 1) identify an unexpected vasoconstrictive effect imparted by administration of a medication, which can signify a dysfunctional vascular state, 2) test and compare the effect of existing vasodilator medications for a specific coronary artery and individual for the purpose of tailoring medical therapy, and 3) test and characterize the unknown effects of medications on coronary artery resistance.

While the preceding descriptions discussed the disclosed subject matter's method of coronary artery resistance analysis, the means of analysis is also worth discussing and considering for patent protection. All the described calculations can be performed using measurements of FFR and CFR, by whatever means is used. Existing methods can measure FFR alone, CFR alone, or even both FFR and CFR simultaneously. Nevertheless, for whatever means of measuring FFR and CFR is used, the subsequent use of the described method to analyze coronary artery resistance is a topic for patent protection. Furthermore, while the disclosed subject matter uses pressure-derived CFR as its means to calculate CFRp, the uniqueness/patentability of the disclosed subject matter's specific means of measuring CFRp should not impact the separate concern regarding the uniqueness/patentability of the disclosed subject matter's subsequent coronary artery resistance analysis.

### Diastolic Pressure-Derived CFR

Disclosed is a method comprising: (a) matching a diastolic ventricular pressure measurement and a diastolic arterial pressure measurement from a same pressure source using a pressure wire; (b) recording real-time telemetry data including measurements of hyperemic and baseline coronary blood flow; (c) automatically identifying maximum and minimum diastolic 1-(ventricular pressure/arterial pressure) values in the real-time telemetry data; and (d) calculating diastolic pressure-derived coronary flow resistance at least in part from the maximum and minimum diastolic 1-(ventricular pressure/arterial pressure) values.

Disclosed is a system used to analyze characteristics of blood flow within arteries supplying heart muscle with blood (coronary arteries). Existing devices are commercially available to provide clinically-important measurements of coronary blood flow, including fractional flow reserve (FFR) and coronary flow reserve (CFR), but feature significant limitations. FFR is highly-useful in evaluating the degree of flow-limiting disease within large (epicardial) coronary arteries, but does not evaluate the condition of distal microscopic blood vessels (microvasculature). Conversely, CFR is used to evaluate microvascular function, but does not quantify epicardial vessel disease. FFR is based on highly-reproducible invasive blood pressure measurements, while CFR calculations using blood velocities or thermodilutional flow rates are limited by poor reproducibility and accuracy. The disclosed system is designed to overcome these limitations by providing a single platform to collect and analyze real-time hemodynamic data, and uniquely report simultaneous FFR and CFR measurements calculated from invasive blood pressure measurements alone.

FFR is a measurement derived from comparing simultaneous invasive blood pressure measurements acquired proximal to (upstream) and distal to (downstream) a segment of diseased coronary artery, and obtained during maximal coronary blood flow (hyperemia). Coronary artery disease (CAD) causes resistance to blood flow, thus increasing distal vessel blood velocity, decreasing distal vessel blood pressure, and increasing the difference between proximal and distal pressure measurements. This phenomenon is consistent with the Poiseuille law explaining the pressure drop of incompressible fluid flowing through a long cylindrical pine of constant cross section. FFR is calculated as the ratio of distal pressure (Pd) to proximal aortic pressure (Pa) during hyperemia, and conceptually represents the proportion of blood flow achieved in the presence of the interrogated coronary artery obstruction compared to without obstruction. Thus, FFR values of 1.0 indicate unobstructed blood flow through an interrogated vessel segment.

In clinical use FFR is arguably the gold-standard for identifying significant coronary artery obstructions and guiding therapy for individuals with CAD. Randomized clinical trials have demonstrated superior patient outcomes with FFR guidance compared to using coronary angiography alone, as well as superiority of coronary artery revascularization compared to medical therapy alone in those with obstructive coronary artery disease defined by FFR ≤0.80 (1-2). Validated variations of FFR include 1) instantaneous wave-free ratio (iFR) that reports resting Pd/Pa ratio specifically during relaxation of the ventricular heart chamber (diastole) when coronary blood flow predominantly occurs, and 2) average resting Pd/Pa measured indiscriminately across the entire cardiac cycle. Both measurements are recorded without inducing hyperemia. Reported FFR, iFR, and Pd/Pa are discrete decimal values ≤1.0.

A similar measurement to FFR is CFR. While FFR compares measurements from two different source locations during an identical coronary flow state, CFR compares measurements from a single source location during two different coronary flow states (hyperemic blood flow versus baseline blood flow). The CFR measurement indicates the ability of the coronary artery microvasculature to maximally dilate and increase coronary artery flow. An abnormal CFR identifies diseased microvasculature warranting medical therapy and/or compensatory dilation in the presence of an upstream obstruction. Currently, there are two ways to measure CFR: 1) changes in blood velocity using ultrasound Doppler, and 2) changes in blood flow rate using the thermodilutional method. “Normal” Doppler-based CFR values ≥2.0 are shown to have high predictive accuracy with non-invasive nuclear myocardial perfusion imaging results in individuals without angiographic evidence of CAD (3). Patients with discordant CFR and FFR measurements, particularly with abnormal CFR <2.0 and normal FFR >0.80, possess greater risk of adverse cardiac events (4), emphasizing the independence of CFR and FFR in measuring different CAD conditions.

### Limitations of Existing Devices

Contemporary guidewires and microcatheters for FFR and Pd/Pa ratio measurements in the clinical setting are available from multiple manufacturers. These pressure wires are commonly used in contemporary cardiovascular procedures, first to interrogate segments of diseased epicardial coronary arteries, and second to deliver therapeutic balloon and stent catheters across regions of obstructive disease during subsequent percutaneous coronary intervention (PCI). Despite their widespread use and practical application during routine PCI, none of these pressure-only devices provide a measurement of CFR.

Compared to traditional “workhorse” wires or the pressure-only wires, Doppler-based wires are relatively unwieldy and challenging to manipulate within the coronary anatomy. As such, they are impractical for routine hemodynamic studies and utilization for PCl despite having regulatory approval for these uses.

In addition to these practical considerations, limitations are further encountered during traditional CFR measurement. Doppler signals at any point of the cardiac cycle can be overestimated due to signal noise or artifacts, or underestimated due to suboptimal wire angulation away from the direction of flow and/or positioning against the vessel wall. Furthermore, optimal signals acquired at baseline flow may deteriorate during hyperemia, or vice versa. Any of these suboptimal Doppler measurements introduces CFR calculation errors. Following an injection of cold saline through a temperature-based device, a thermodilution curve is created over multiple heart beats and used to derive coronary blood flow. Thermodilutional CFR measurement can be poorly reproducible, user dependent, and time-consuming. This technique also requires intravenous adenosine infusion (rather than an intracoronary adenosine bolus) to induce continuous hyperemia for minutes at a time, which not only allows multiple temperature curves to be acquired during both hyperemic and baseline flow states, but also prolongs diagnostic procedures. Description of the disclosed subject matter

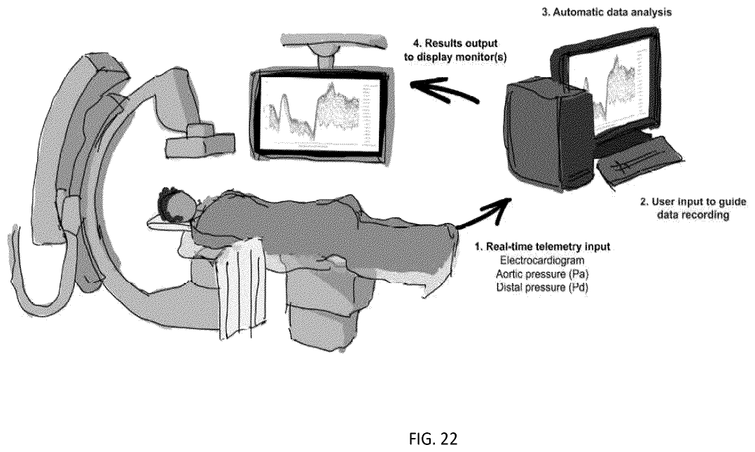

The disclosed system features both 1) a physical device that receives real-time patient telemetry data and user input to guide data recording, and 2) data analysis algorithms to create the unique measurement of diastolic pressure-derived CFR (CFRp) that is then reported to the user (FIG. 22).

The disclosed system's physical device receives patient telemetry data including real-time, simultaneous Pa, Pd, and electrocardiographic waveforms. Pressure data are typically obtained from commercially-available devices positioned within aorta (Pa) and distal coronary artery (Pd) during invasive cardiovascular procedures. A fluid-filled guiding catheter positioned at the coronary artery ostium provides Pa measurements, while second manometer (integrated on a small wire or catheter) is advanced into a distal coronary artery segment (FIG. 2). These data are routinely measured by commercially-available devices every 4 to 5 milliseconds. The disclosed system is guided by user input to record and automatically processes data, discussed below. The device then exports analyzed data to a display monitor for user review.

The disclosed system's data analysis functions include 1) baseline matching of diastolic Pa and Pd measurements from the same pressure source, 2) measurement of beat-by beat diastolic 1-Pd/Pa when inducing hyperemia, 3) measurement of diastolic 1-Pd/Pa at coronary flow baseline, and 4) calculation of diastolic CFRp. Each step is described in more detail below.

### Disclosed Algorithm Step 1: Pressure Matching

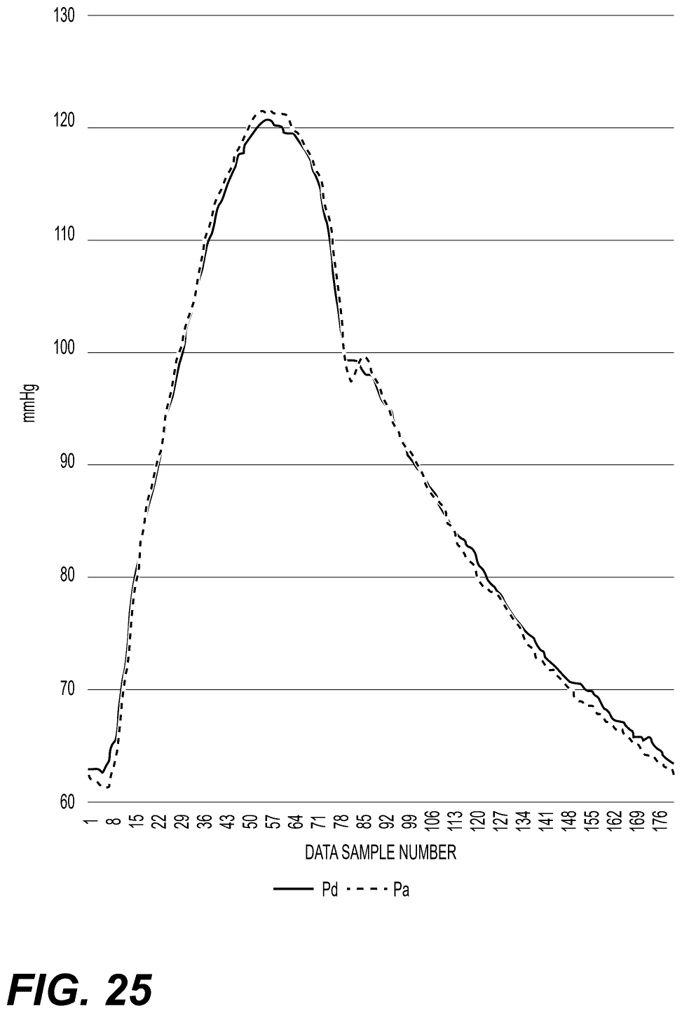

Baseline matching of Pa and Pd measurements acquired from the same sampling location (also called “equalization” or “normalization” for commercially-available products) is routinely performed immediately prior to performing standard FFR measurements. This is accomplished by positioning the pressure wire/catheter at the tip of the guiding catheter to compare measurements acquired from the same pressure source (FIG. 24). Commercially-available devices compare Pa and Pd pressure averages (measured indiscriminately across various parts of the cardiac cycle), and correct one waveform mean to match the other (FIG. 25). The disclosed system will specifically make appropriate waveform corrections to ensure matching of diastolic pressures rather than indiscriminately doing so across non-specific parts of the cardiac cycle. The disclosed system's method will also record data during this equalization process to allow retrospective data correction.

### Disclosed Algorithm Steps 2 and 3: Measurement of Beat-by-Beat Diastolic 1-Pd/Pa During Hyperemic and Baseline Coronary Blood Flow

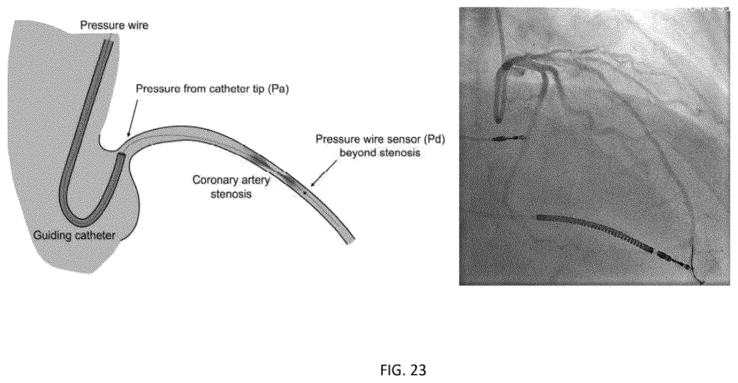

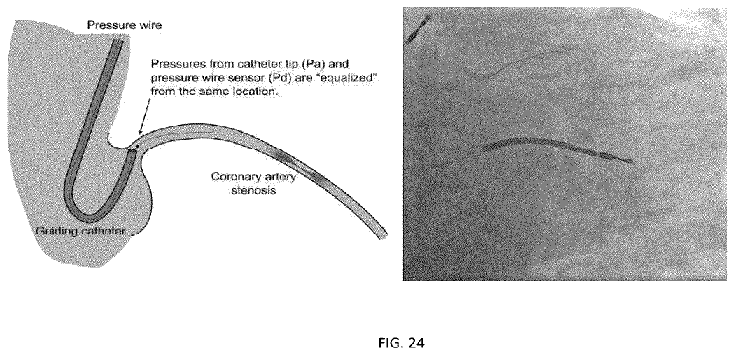

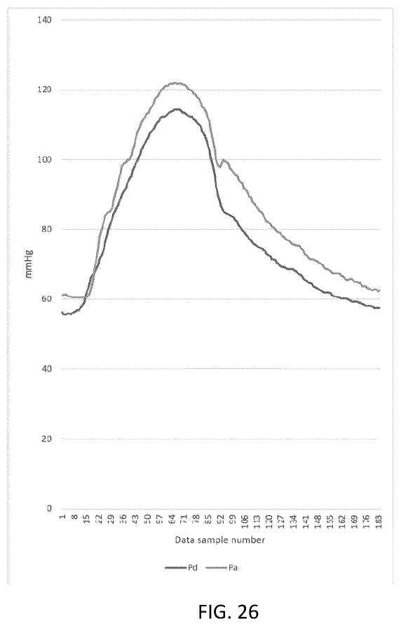

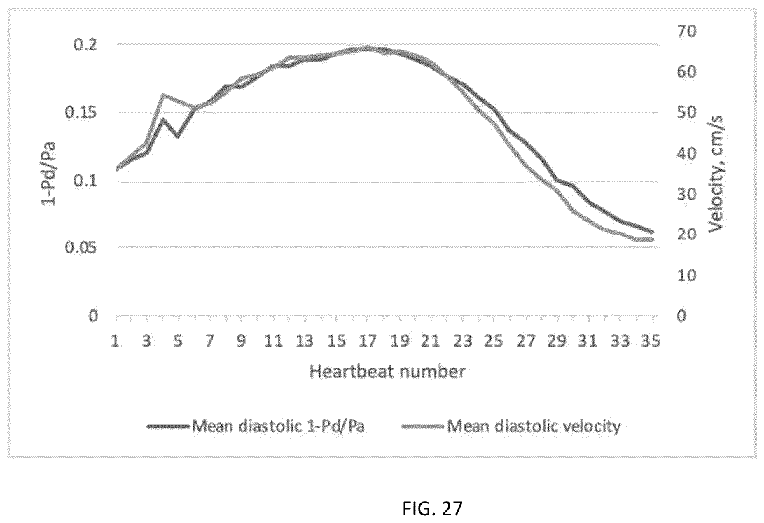

Following step 1, the pressure wire/catheter is then advanced by the user into a distal part of the coronary artery anatomy (FIG. 23). In the presence of anatomical resistance to blood flow between the two pressure sources (e.g., CAD), a decrease in Pd is observed compared to the reference Pa (FIG. 26). The disclosed subject matter then calculates the unique value “1-Pd/Pa” during the diastolic period of each heartbeat. This value conceptually represents the proportion of resistance due to epicardial vessel anatomy between the two pressure sources, compared to total coronary artery resistance of the entire interrogated vessel (entire epicardial vessel plus distal microvasculature). Minimum diastolic 1-Pd/Pa is observed during baseline coronary flow (when microvascular resistance is greatest), and maximum diastolic 1-Pd/Pa is observed during hyperemia (when microvascular resistance is minimal due to adenosine-induced vasodilation) (FIG. 27). The ratio of diastolic 1-Pd/Pa occurring during hyperemia versus baseline yields values ≥1.0, and is a unique calculation of diastolic CFRp reported by the disclosed system.

The disclosed process of calculating a diastolic CFRp value begins with accepting user input to start and stop a recording of real-time telemetry data to include both baseline and hyperemic coronary blood flow (e.g., data following an intracoronary bolus or intravenous infusion of adenosine). From this recording, the disclosed system automatically calculates aggregated diastolic 1-Pd/Pa (e.g., mean, median, or other) for each heartbeat. Heartbeats with significant artifacts are automatically excluded from analysis. Maximum and minimum diastolic 1-Pd/Pa values are automatically identified within the recording.

### Disclosed System Algorithm Step 4: Calculation of Diastolic CFRp

One the disclosed system identifies maximum and minimum diastolic 1-Pd/Pa values, it automatically calculates the maximum/minimum ratio. The resulting value, diastolic CFRp, is then reported by the disclosed subject matter to the user based on the following equations:

$$diastolic CFRp = \frac{{( 1 - \frac{Pd}{P a} )}_{hyperemia , diastole}}{{( 1 - \frac{Pd}{P a} )}_{hyperemia , diastole}} \qquad ( equation 1 )$$

$$diastolic CFRp = \frac{{( \frac{Pd - P a}{P a} )}_{hyperemia , diastole}}{{( \frac{Pd - P a}{P a} )}_{hyperemia , diastole}} \qquad ( equation 2 )$$

Multiple commercially-available devices routinely calculate and report Pd/Pa averaged indiscriminately across various parts of the cardiac cycle. For example, a device can identify pressures obtained during the diastolic period and report an iFR measurement, which is a unique, commercially-available measurement specifically obtained without inducing a hyperemic flow state. The purpose of this iFR measurement is specifically to serve as an alternative to FFR without having to induce a hyperemic flow state.

For example, the system performs an analysis compared to the commercially-available Pd/Pa, iFR, and FFR measurements described above. First, the disclosed subject matter measures the unique value 1-Pd/Pa of each heartbeat that no other commercially-available device measures (equation 1). The formula can be rearranged as (Pa−Pd)/Pa and represents an instantaneous pressure gradient across the interrogated coronary artery segment, which is then standardized by Pa pressure at time of gradient measurement (equation 2). Commercially-available devices report neither 1-Pd/Pa, nor its equivalent (Pa Pd)/Pa, measurements.

Second, the system calculates its unique measurements during the diastolic period. iFR measurement is calculated using a fundamentally different equation (diastolic Pd/Pa at baseline flow state alone) and evaluates a different hemodynamic property (epicardial vessel obstruction) compared to CFRp (microvascular function).

Third, the system performs repeated measurements across multiple heartbeats to identify 1-Pd/Pa values during both baseline flow and hyperemia. While existing pressure-based measures are acquired during either hyperemia or baseline flow alone, none uses both flow states in its calculation. In particular, the iFR measurement differs from CFRp not only by the equation on which it is based, but also its intentional use of data acquired during the baseline coronary flow state alone.

Fourth, the system's diastolic CFRp measurement resembles, but is not identical to, traditional CFR measurement. The key difference lies in data collected during the diastolic period (disclosed subject matter) versus the entire cardiac cycle (traditional CFR). Thus, reported normal range for traditional CFR ≥2.0 does not apply to CFRp. The normal range for diastolic CFRp in those without CAD will need to be identified independently.

### Comparison of the Disclosed System to Other Proposed Versions of CFRp

Previous attempts to calculate and/or validate versions of CFRp have been made and have been hindered by poor correlation with established traditional CFR measurements (Doppler or thermodilution methods) or adverse cardiac events. The following calculations have been previously described:

$$CFR = \sqrt{\frac{\overline{P} a - \overline{P} {d}_{hyperemia}}{\overline{P} a - \overline{P} {d}_{Rest}}} \qquad ( equation 3 )$$

$$CFR = \sqrt{\frac{P a - {Pd}_{hypermia max value}}{P a - {Pd}_{rest max value}}} \qquad ( equation 4 )$$

$$CFR = \frac{\overline{P} a - \overline{P} {d}_{hyperemia}}{\overline{P} a - \overline{P} {d}_{rest}} \qquad ( equation 5 )$$

Fundamental differences are observed between these versions of CFRp (equations 3-5) and those of the disclosed subject matter (equations 1-2). Equations 3 and 4 most notably use a square root function to transform a ratio of simple pressure gradients. Equation 5 uses a ratio of simple pressure gradients alone. Equations 3 and 5 use mean data from across the entire cardiac cycle while equation 4 uses single maximum gradient values that may or may not occur during diastole. No equation uses either 1) the ratio of 1-Pd/Pa values (or its rearranged version [Pd−Pa]/Pa), or 2) dedicated use of aggregated diastolic pressure data, which are among the key distinguishing components of the disclosed subject matter.

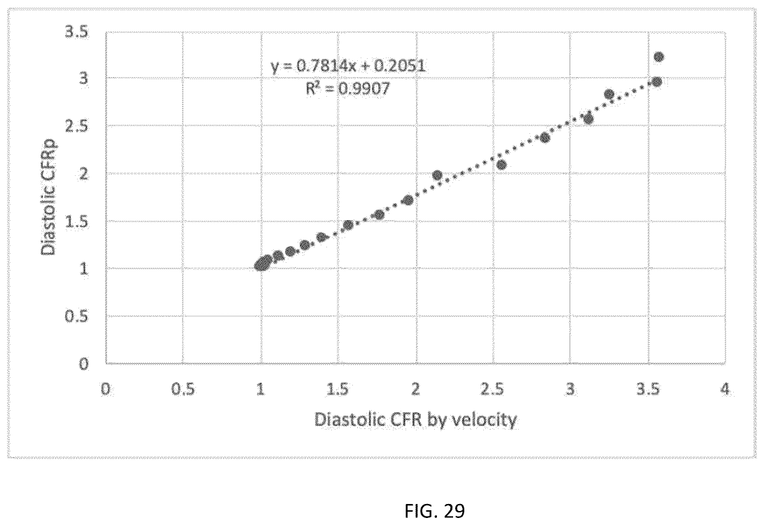

These distinguishing features of the disclosed subject matter are intentional and critical to creating a clinically-useful analog of traditional CFR. First, beat-by-beat pressure-derived 1-Pd/Pa and high-quality Doppler-based blood velocity tracings following adenosine-induced hyperemia are strikingly similar during diastole, and dissimilar during systole (FIG. 7). The invention capitalizes on the similarity by including diastole, and excluding systole, in its data selection and calculations (contrary to equations 3 and 5). Tracing fluctuations during diastole are normalized by aggregating data during the diastolic period (contrary to single maximum value used in equation 4). Second, beat-by-beat CFR calculations derived from 1-Pd/Pa and Doppler velocity ratios have a very strong positive linear, rather than square root, relationship (contrary to equations 3 and 4) (FIG. 29).

### Clinical Implications

The disclosed subject matter's ability to calculate diastolic pressure-derived CFR enables a single pressure wire/catheter to simultaneously quantify disease affecting both large-caliber coronary arteries and its distal microvasculature. With contemporary pressure-only devices, this dual evaluation is not possible. The disclosed subject matter aims to transform the existing “golf-standard” FFR technique, already highly recommended by clinical guidelines for optimal patient management (10), into a tool that provides twice as much diagnostic information as currently possible. Its pressure-based design also overcomes the inherent limitations of traditional CFR, making CFRp measurements as rapid, accurate, and reproducible as FFR. As a result, it can identify individuals with significant microvascular disease who may otherwise have “normal” FFR results that delay appropriate therapy and conceal risk of adverse clinical outcomes. The disclosed system also obviates the need for ultrasound Doppler or thermodilution guidewires that are suboptimal for routine clinical use, especially true when transitioning from an abnormal diagnostic study to PCL. Overall, this disclosed subject matter expands the diagnostic utility of every FFR procedure performed in cardiac catheterization laboratories worldwide and provides a useful new measurement of microvascular disease.

FIG. 22 illustrates general features of the disclosed subject matter and interface within a cardiac catheterization laboratory.

FIG. 23 illustrates a typical setup for collection of simultaneous aortic blood pressure (Pa) and distal coronary artery pressure (Pd) during traditional FFR measurements, as well as use of the disclosed subject matter for CFRp measurements (left image). The angiogram shows a guiding catheter engaging a left coronary artery and a pressure wire positioned in the distal segment of a left anterior descending artery (right image).

FIG. 24 illustrates a typical guiding catheter and pressure wire arrangement to simultaneously measure aortic pressure (Pa) and distal pressure (Pd) from the same pressure source (tip of the guide catheter) during pressure “equalization” (left image). The angiogram shows a guiding catheter engaging a left coronary artery and pressure wire sensor positioned at the tip of the catheter during pressure “equalization” (right image)

FIG. 25 illustrates simultaneous Pa and Pd measurements during one heartbeat immediately following commercially-available pressure “equalization.” Mean pressures calculated across the entire heartbeat are identical (87 mmHg), while peak systolic and end diastolic pressures (data sample 110 to end) are not.

FIG. 26 illustrates that distal intracoronary pressure measurements (Pd) are reduced compared to the guiding catheter reference measurements (Pa) in the presence of anatomical resistance between the two pressure sources.

FIG. 27 illustrates simultaneous beat-by-beat calculation of mean diastolic 1-Pd/Pa and mean diastolic velocity following an intracoronary bolus of adenosine to induce hyperemia. Peak values of each occurs at hyperemia, while minimum values occur when coronary flow returns baseline.

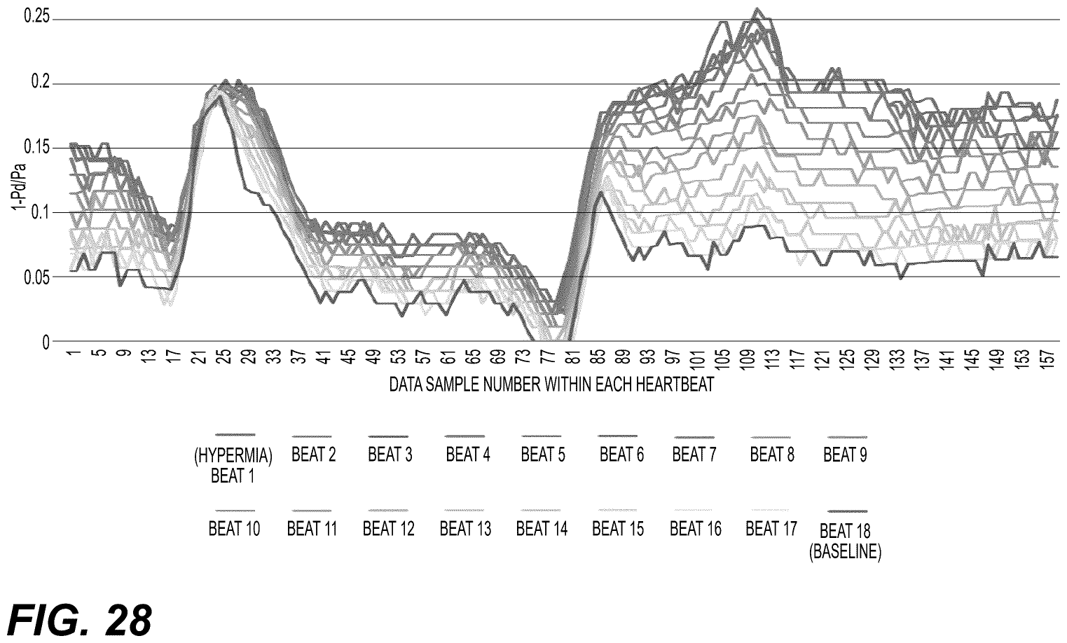

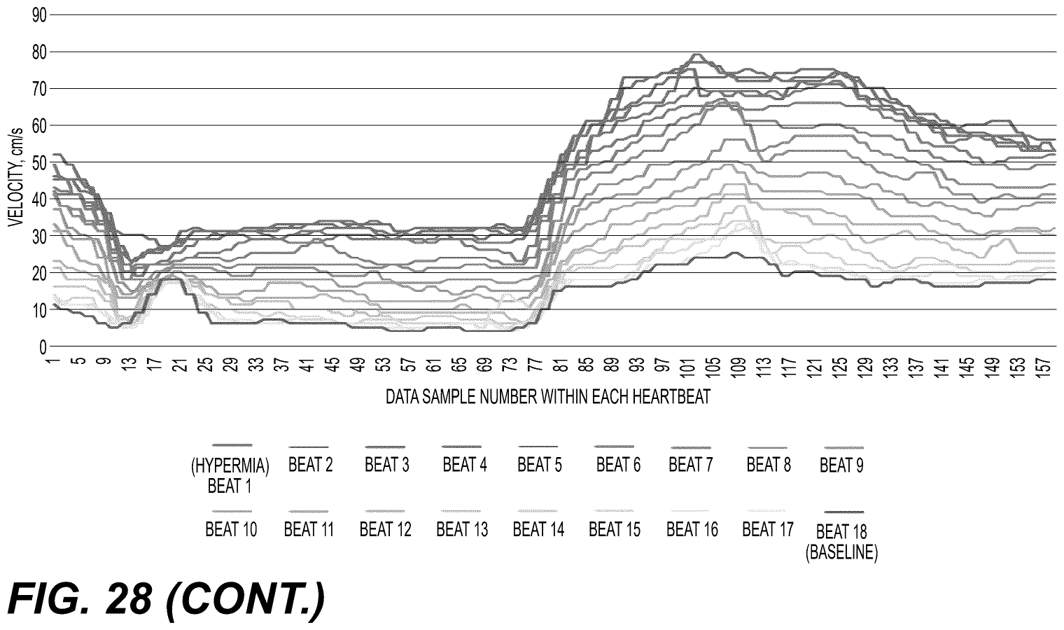

FIG. 28 illustrates exemplary beat-by-beat comparison of pressure-derived 1-Pd/Pa (top graph) versus Doppler-derived blood velocity (bottom graph) following adenosine-induced hyperemia and return to baseline coronary flow. Tracings are most similar during diastole of each heartbeat (approximate data sample numbers 100 to end) as peak hyperemic values (2810) decrease in a stepwise fashion back to baseline (2820). Significant dissimilarities are observed during early systole and late systole (top graph peak and trough).

FIG. 29 illustrates exemplary beat-by-beat comparison of diastolic pressure-derived CFR (CFRp) versus diastolic Doppler-based CFR following adenosine-induced hyperemia and return to baseline coronary flow. Diastolic CFR and CFRp data have a very strong positive linear correlation (R²=0.991, P-value <0.001). Deviation of the relationship slope from 1.0 may be the result of systematic bias of either Doppler or pressure values.

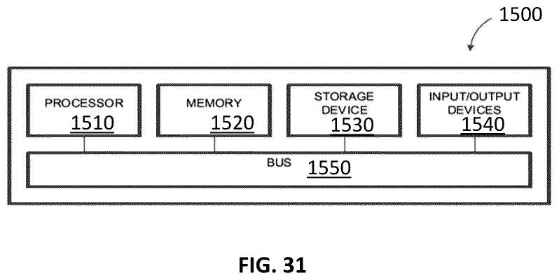

FIG. 31 depicts a block diagram illustrating a computing system 1500 consistent with implementations of the current subject matter. For example, the computing system 1500 can be used to implement analysis of a pressure loop plot and/or any components therein.

As shown in FIG. 31, the computing system 1500 can include a processor 1510, a memory 1520, a storage device 1530, and input/output device 1540. The processor 1510, the memory 1520, the storage device 1530, and the input/output device 1540 can be interconnected via a system bus 1550. The processor 1510 is capable of processing instructions for execution within the computing system 1500. Such executed instructions can implement one or more components of, for example, a model for analyzing a pressure loop plot. In some example implementations, the processor 1510 can be a single-threaded processor. Alternatively, the processor 5110 can be a multi-threaded processor. The processor 1510 is capable of processing instructions stored in the memory 1520 and/or on the storage device 1530 to display graphical information for a user interface provided via the input/output device 1540.

The memory 1520 is a computer readable medium such as volatile or non-volatile that stores information within the computing system 500. The memory 1520 can store data structures representing configuration object databases, for example. The storage device 1530 is capable of providing persistent storage for the computing system 1500. The storage device 1530 can be a floppy disk device, a hard disk device, an optical disk device, a tape device, a solid-state device, and/or any other suitable persistent storage means. The input/output device 1540 provides input/output operations for the computing system 1500. In some example implementations, the input/output device 1540 includes a keyboard and/or pointing device. In various implementations, the input/output device 1540 includes a display unit for displaying graphical user interfaces.

According to some example implementations, the input/output device 1540 can provide input/output operations for a network device. For example, the input/output device 1540 can include Ethernet ports or other networking ports to communicate with one or more wired and/or wireless networks (e.g., a local area network (LAN), a wide area network (WAN), the Internet).

In some example implementations, the computing system 1500 can be used to execute various interactive computer software applications that can be used for organization, analysis and/or storage of data in various formats. Alternatively, the computing system 1500 can be used to execute any type of software applications.

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures. and/or described herein do not necessarily require the particular order shown, or sequential order, to achieve desirable results. For example, the logic flows may include different and/or additional operations than shown without departing from the scope of the present disclosure. One or more operations of the logic flows may be repeated and/or omitted without departing from the scope of the present disclosure. Other implementations may be within the scope of the following claims.

1-75. (canceled)

76. A method of characterizing cardiovascular blood pressurization, comprising:
acquiring measurements of cardiovascular activity within a ventricle during at least one entire cardiac cycle;
generating, based on the acquired measurements, at least one pressure loop representing the change in ventricular pressure over time relative to the pressure;
determining at least one pressure loop characteristic, wherein the at least one pressure loop characteristic is one or more of loop size, loop shape symmetry, loop shape roughness, or a number of loop shape indentations; and
displaying a graphic to a user indicating the at least one determined pressure loop characteristic.

77. The method of claim 76, wherein the acquired measurements comprise raw pressure data.

78. The method of claim 76, wherein determining the at least one pressure loop characteristic comprises computing one or more loop parameters including loop area, a loop shape, a loop cycle duration, characteristics of a top border of the at least one pressure loop, characteristics of a bottom border of the at least one pressure loop, characteristics of a left border of the at least one pressure loop, characteristics of a right border of the at least one pressure loop, and timing of loop features.

79. The method of claim 78 further comprising comparing the one or more loop parameters to a reference value of the one or more loop parameters to identify a cardiovascular anomaly.

80. The method of claim 78, wherein determining the at least one pressure loop characteristic further comprises comparing a plurality of the one or more loop parameters.

81. The method of claim 76, wherein the acquired measurements of cardiovascular activity within the ventricle are acquired during a plurality of entire cardiac cycles.

82. The method of claim 78, wherein the characteristics of the left border of the at least one pressure loop comprises a pre-a wave diastolic pressure.

83. The method of claim 82, wherein the pre-a wave diastolic pressure is determined as a pressure within the ventricle when the third derivative of the ventricular pressure over time is positive.

84. The method of claim 78, wherein determining the at least one pressure loop characteristic further comprises comparing two or more loop parameters at different time points.

85. The method of claim 83, wherein the one or more loop parameters comprise characteristics of the left border of the at least one pressure loop and loop area, and the method further comprises computing a relationship, across the plurality of cardiac cycles, between ventricular end-diastolic pressure and loop area, the ventricular end-diastolic pressure being determined from the characteristics of the left border of the at least one pressure loop.

86. The method of claim 83, wherein the one or more loop parameters comprise characteristics of the left border of the at least one pressure loop and characteristics of the right border of the at least one pressure loop, and the method further comprises computing a relationship, across the plurality of cardiac cycles, between ventricular end-diastolic pressure and maximum ventricular pressure, the ventricular end-diastolic pressure being determined from the characteristics of the left border of the at least one pressure loop and the maximum ventricular pressure being determined from the characteristics of the right border of the at least one pressure loop.

87. The method of claim 76, wherein determining the at least one pressure loop characteristic comprises computing ventricular power.

88. The method of claim 87, wherein determining the at least one pressure loop characteristic comprises computing one or more of downstream blood flow, ventricular power, ventricular resistance, elasticity, compliance, contractility, or stroke volume.

89. The method of claim 76, wherein processing the at least one pressure loop is performed by a machine learning model.

90. The method of claim 76, further comprising acquiring measurements of cardiovascular activity within a second ventricle during the at least one entire cardiac cycle;
generating, based on the acquired measurements, at least one pressure loop representing the change in ventricular pressure over time relative to the pressure;
determining at least one pressure loop characteristic corresponding to the at least one pressure loop characteristic determined for the ventricle; and
displaying a graphic to a user indicating the at least one determined pressure loop characteristics.

91. A method of characterizing cardiovascular blood pressurization, comprising:
acquiring measurements of cardiovascular activity within a ventricle of a subject during at least one entire cardiac cycle;
generating, based on the acquired measurements, at least one pressure loop representing the change in ventricular pressure over time relative to the pressure;
determining at least one pressure loop characteristic, wherein the at least one pressure loop characteristic is one or more of loop area, loop shape symmetry, loop shape roughness, or a number of loop shape indentations;
comparing the at least one determined pressure loop characteristic to a reference pressure loop characteristic; and
treating the subject based on the comparison, wherein the comparison indicates the subject is impaired by one or more of coronary artery disease, ischemic cardiomyopathy, global non-ischemic cardiomyopathy, or hypertrophic cardiomyopathy.

92. The method of claim 91, wherein the at least one determined pressure loop characteristic comprises loop shape roughness, and when the loop shape roughness satisfies a reference loop shape roughness, the comparison indicates the subject is impaired by coronary artery disease and/or ischemic cardiomyopathy.

93. The method of claim 91, wherein the at least one determined pressure loop characteristic comprises loop shape roughness and loop size, and when the loop shape roughness satisfies a reference loop shape roughness and the loop size satisfies a reference loop size, the comparison indicates the subject is impaired by global non-ischemic cardiomyopathy.

94. The method of claim 91, wherein the at least one determined pressure loop characteristic comprises loop shape roughness, and when the loop shape roughness satisfies a reference loop shape roughness, the comparison indicates the subject is impaired by hypertrophic cardiomyopathy.

95. A method of characterizing cardiovascular blood pressurization, comprising:
acquiring measurements of cardiovascular activity within a ventricle during at least one entire cardiac cycle;
generating, based on the acquired measurements, at least one pressure loop representing the change in ventricular pressure over time relative to the pressure; and
displaying the at least one pressure loop as a graphic to a user.
